(19) United States
(12) Patent Application Publication
(10) Pub. No.: US 2018/0140536 A1
(43) Pub. Date: May 24, 2018

(54) IVORY NUT POWDER AND MANNAN FROM IVORY NUT

(71) Applicant: EPC Natural Products Co., Ltd., Beijing (CN)

(72) Inventors: Jing Li, Beijing (CN); Jingang Shi, Beijing (CN); Jian Du, Beijing (CN); Ming Li, Beijing (CN)

(21) Appl. No.: 15/797,120

(22) Filed: Oct. 30, 2017

Related U.S. Application Data
(60) Provisional application No. 62/425,129, filed on Nov. 22, 2016.
(60) Provisional application No. 62/565,322, filed on Sep. 29, 2017.

(51) Int. Cl. A61K 8/9794; A61Q 17/04; A61K 8/73; A61K 36/889; A61K 31/715; A61Q 11/00; A61Q 1/02; A61Q 1/12; A61Q 19/00; A61Q 1/10; A61Q 15/00; A61K 9/14; A61K 8/02; A61K 8/25; A61K 8/29; A23L 33/105

(52) U.S. Cl. CPC A61K 8/9794; A23V 2002/00; A61K 8/73; A61K 36/889; A61K 31/715; A61Q 11/00; A61Q 1/02; A61Q 1/12; A61Q 19/007; A61Q 1/10; A61Q 15/00; A61K 9/14; A61K 8/022; A61K 8/025; A61K 8/0245; A61K 8/25; A61K 8/29; A61K 8/732; A23L 33/105; A61K 2800/28; A61Q 17/04

(57) ABSTRACT

The invention describes ivory nut and/or hydrolyzed ivory nut (mannan) compositions that are provided as fine particles that can be used in various cosmetic compositions, such as exfoliating compositions, as well as in toothpastes, body washes, creams and lotions, sun screens, make up, baby powders and the like.

### CROSS REFERENCE TO RELATED APPLICATIONS

This application claims the priority dates of U.S. Provisional Patent Application Ser. No. 62/425,129, filed Nov. 22, 2016 and Provisional Patent Application Ser. No. 62/565,322, filed Sep. 29, 2017, the disclosures of which are incorporated by reference herein in their entirety.

### FIELD OF THE INVENTION

The invention relates generally to ivory nut and/or mannan particulates that are useful in various compositions, such as cosmetics, body scrubs, sun screen and the like. The particulate described herein can be bleached or dyed with various colors.

### BACKGROUND OF THE INVENTION

Particles from plants, minerals or microplastics are utilized in a variety of personal care products, including use as exfoliating agents.

Stone containing fruits, such as apricot, almond, walnut, cherry, olives, etc. are used in scrubs in the cosmetic industry as they tend to be cost-effective. However, their typical dark color often causes the cosmetic product to become darkened which is not always visually appealing to the consumer.

Mineral particles, such as pumice, sand (silica), talc, titanium dioxide, and other minerals, can be white in color but are often too abrasive for cosmetic and/or exfoliating purposes. They also tend to have a high density (volumetric mass) which requires changes in formulation to keep the particles suspended in the product.

Microplastics are used extensively in personal care products for exfoliation purposes. The microbeads used in personal care products are mainly made of polyethylene (PE), but can also be made of polypropylene (PP), polyethylene terephthalate (PET), polymethyl methacrylate (PMMA) or nylon. In recent years, the potentially negative impact of microplastics on the environment has been explored and this has led to the decision by some cosmetic companies to remove microplastics from their formulations.

After usage, they pass through the sewage treatments plants without being filtered or are washed down the drain directly. Either way, they end up in rivers, lakes and oceans resulting in water pollution since they are not biodegradable.

This has now being addressed by several authorities all over the world, which require or strongly recommend the plastic microbeads be phased out and substituted by other more adequate substances. Bans for plastic microbeads have been implemented and are quickly due, e.g. by end of 2016 in the Netherlands and starting on Jul. 1, 2018 in the United States as they negatively impact on the environment.

Amorphous form silica is applied in personal care, food products, and other industries. Although silica is permitted as a food additive and as an ingredient in personal care products in a limited amount, the concerns of health hazards from consumers have been increasing for years.

For example, health effects from silica exposure can include, silicosis, a disabling, non-reversible and sometimes fatal lung disease; other non-malignant respiratory diseases, such as chronic bronchitis; lung cancer; and/or kidney disease, including nephritis and end-stage renal disease.

Talcum powder is made from talc, a mineral that includes magnesium, silicon, and oxygen. As a powder, talc absorbs moisture well and helps cut down on friction, making it useful for keeping skin dry and helping to prevent rashes. It is widely used in cosmetic products such as baby powder and adult body and facial powders, as well as in a number of other consumer products.

In its natural form, some talc contains asbestos, a substance known to cause cancers in and around the lungs when inhaled. All talcum products used in homes in the United States have been asbestos-free since the 1970s.

Most concerns about a possible link between talcum powder and cancer have focused on whether people who have long-term exposure to natural talc fibers at work, such as talc miners, are at higher risk of lung cancer from breathing them in and whether women who apply talcum powder regularly in the genital area have an increased risk of ovarian cancer.

Millions of tons of titanium dioxide are produced globally each year. It adds whiteness and brightness to products and also helps them resist discoloration. Titanium dioxide also reflects ultraviolet (UV) light, which is why it is often used as an ingredient in sunscreens.

Most titanium dioxide (close to 70 percent) is used as a pigment in paints, but it is also added to cosmetics, toothpastes, pharmaceuticals, paper and food.

Titanium dioxide is generally considered to be a relatively inert, safe material, but an increasing number of products are now using titanium dioxide nanoparticles, and that may change everything. Nanoparticles are ultramicroscopic in size, making them able to readily penetrate your skin and travel to underlying blood vessels and your bloodstream. Evidence suggests that some nanoparticles may induce toxic effects in your brain and cause nerve damage, and some may also be carcinogenic. The International Agency for Research on Cancer (IARC) classifies titanium dioxide as a Group 2B carcinogen, which means it is “possibly carcinogenic to humans.” This was based on an animal study showing inhaling high concentrations of titanium dioxide dust may lead to lung cancer.

Further, animal studies indicate significant accumulation of nanoparticles in the brain, while toxicity studies have shown the particles have negative effects on brain cell viability and function. One recent study even showed titanium dioxide nanoparticles induced “an increase in reactive oxygen species generation, and a decrease in mitochondrial membrane potential, suggesting mitochondrial damage.” The researchers believe exposure to the particles may lead to neurological dysfunction. Specifically, the nanoparticles were found to harm astrocyte cells, which help regulate serotonin, dopamine and other neurotransmitters.

Other research also suggests titanium dioxide nanoparticles may have hidden risks for brain health. For instance: prenatal exposure to titanium dioxide nanoparticles may result in alteration to the cerebral cortex, olfactory bulb and brain regions intimately related to dopamine systems of offspring mice. Exposure to titanium dioxide nanoparticles may alter oxidative and inflammatory responses as well as the renin-angiotensin system in the brain (which plays a role in cardiovascular health, including hypertension, and aging), thereby modulating brain function. Titanium dioxide nanoparticles induce strong oxidative stress and mitochondrial damage in glial cells in the brain. According to research published in Free Radical Biology & Medicine, titanium oxide nanoparticles can enter directly into the brain through the olfactory bulb and can be deposited in the hippocampus region . . . and they were found to produce morphological changes, damage of mitochondria, and an increase in mitochondrial membrane potential, indicating toxicity.

Therefore, a need exists for materials that overcome one or more of the current disadvantages noted above.

### BRIEF SUMMARY OF THE INVENTION

The present invention surprisingly provides ivory nut and/or mannan (the extract or hydrolysis produce from ivory nut) compositions that are provided as fine particles that can be used in various cosmetic compositions, such as exfoliating compositions, as well as in toothpastes, body washes, creams and lotions, sun screens, make up, baby powders, food, feed, pharmaceuticals and the like.

Phytelephas is a genus containing six known species of palms (family Arecaceae), occurring from southern Panama along the Andes to Ecuador, Bolivia, Colombia, northwestern Brazil, and Peru. They are commonly known as ivory palms, ivory-nut palms or tagua palms. The scientific name Phytelephas means “plant elephant”. This refers to the very hard white endosperm of their seeds (tagua nuts or jarina seeds), which resembles elephant ivory. Names include vegetable ivory, palm ivory, marfim-vegetal, corozo, tagua, or jarina. All are included herein and are referred to as “ivory nut”.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 is a photomicrograph of an embodiment of ivory nut powder (200 mesh).

FIG. 2 is another photomicrograph of the same embodiment of FIG. 1 of ivory nut powder (200 mesh) at a higher magnification.

FIG. 3 is another photomicrograph of the same embodiment of FIG. 1 of ivory nut powder (200 mesh) at a higher magnification.

FIG. 4 is a photomicrograph of an embodiment of mannan powder (300 mesh) at 20 microns.

FIG. 5 is a photomicrograph of the same embodiment of FIG. 4 of mannan powder (300 mesh) at 10 microns.

FIG. 6 is a photomicrograph of the same embodiment of FIG. 4 of mannan powder (300 mesh) at 6 microns.

FIG. 7 shows reflectivity of ivory nut powder (95% through 100 mesh sieve).

FIG. 8 shows reflectivity of ivory nut powder (95% through 80 mesh sieve).

FIG. 9 shows the reflectivity of titanium dioxide powder (95%≤50 micrometers).

FIG. 10 shows the reflectivity of titanium dioxide.

FIG. 11 shows the reflectivity of mannan.

FIG. 12 shows the differences between the reflectivity of titanium dioxide and mannan.

FIG. 13 shows the transmittance of different content ratio of titanium dioxide at 200 nm, 300 nm and 400 nm.

FIG. 14 shows the transmittance of different content ratio of DRY-FLO PURE at 200 nm, 300 nm and 400 nm.

FIG. 15 shows the transmittance of different content ratio of mannan at 200 nm, 300 nm and 400 nm.

FIG. 16 shows the transmittance of different content ratio of micronized talc at 200 nm, 300 nm and 400 nm.

FIG. 17 shows the 400 nm transmittance of titanium dioxide, DRY-FLO PURE, mannan and micronized talc with different content ratio.

FIG. 18 shows the 300 nm transmittance of titanium dioxide, DRY-FLO PURE, mannan and micronized talc with different content ratio.

FIG. 19 shows the 200 nm transmittance of titanium dioxide, DRY-FLO PURE, mannan and micronized talc with different content ratio.

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Tagua Nut is the seed from a genus of palm trees known as Phytelephas macrocarpa. These palms are indigenous to the tropical rainforests of South America and can live up to 180 years. After about 10 years the Tagua palm produces its fruit for the first time.

The fruit, also known as mococha, is the size of a grapefruit and before it matures the nut consists of a tasteless crystalline liquid. When the nuts ripen, they fall to the ground and the milky substances transforms to a gelatinous viscous consistency which finally becomes a hard product, similar in color, appearance and feel to natural ivory.

Thus, tagua nuts are sustainable and provides a way to protect endangered rainforests. Additionally, since the seeds are collected or hand-picked from the ground after falling from the trees after they have fully matured and have hardened. Therefore, the trees are not touched or harmed in any way. These environmentally friendly vegetable ivory products help in the conservation of the planet's biodiversity since all the trees in the concerned rainforests remain unaffected.

Furthermore, the harvesting of the nuts helps to protect endangered rain forests as a result of the economic value which is created in a sustainable manner. Collecting of the nuts supports the local economy, provides jobs and also may help to save elephants and other ivory bearing animals.

There are two aspects to the present embodiments. One is directed to the native ivory nut without further processing other than grinding/pulverizing the nut into a particulate that is useful in the embodiments described herein. The second aspect is directed to hydrolyzed ivory nut in the form of mannan. Mannan is a plant polysaccharide that is a linear polymer of the sugar mannose. Plant mannans have β(1-4) linkages. It is a form of storage polysaccharide. Ivory nut is a source of mannan.

Mannan may also refer to a cell wall polysaccharide found in yeasts. This type of mannan has a α(1-6) linked backbone and α(1-2) and α(1-3) linked branches. It is serologically similar to structures found on mammalian glycoproteins. Detection of mannan leads to lysis in the mannan-binding lectin pathway. This mannan is the source of mannan oligosaccharide-based nutritional supplements (MOS) used as prebiotics in animal husbandry and nutritional supplements. The mannan described herein is also ground/pulverized into particulates useful in the embodiments described herein.

Mannan can be produced by alkali hydrolysis or extract of ivory nut with a metal hydroxide, such as sodium hydroxide, in water at 25° C. or less with agitation. The process may be repeated several times and/or the materials may be allowed to be in contact for an extended period of time (e.g., 12 hours) followed by filtration of solids. Solids are collected by filtration, dried and then ground into desired particulate form.

The molecular weight range of the mannan is from about 1800 Da to about 6,000,000 Da with a degree of polymerization (DP) from about 10 to about 30,000.

The term “particulate” is used throughout and is intended to refer to both ivory nut and mannan compositions.

The particulates described herein that are useful in various products/embodiments can have a particle size of less than 4 mesh, less than 6 mesh, less than 8 mesh, less than 12 mesh, less than 16 mesh, less than 20 mesh, less than 30 mesh, less than 40 mesh, less than 50 mesh, less than 60 mesh, less than 70 mesh, less than 80 mesh, less than 100 mesh (about 149 microns), less than 140 mesh, less than 200 mesh (less than 74 microns), less than 1000 microns, less than 500 microns, less than 200 microns, less than 100 microns, less than 50 microns, less than 20 microns, less than 10 microns, less than 5 microns, less than 1 micron or less than 0.2 microns.

The particulates can be spherical, elliptical, irregular in form, in shards or mixtures thereof and can be amorphous or crystalline or mixtures thereof.

The ivory nut powder and or mannan of the compositions can make up 1% wt/wt, 2% wt/wt, 3% wt/wt, 4% wt/wt, 5% wt/wt, 6% wt/wt, 7% wt/wt, 8% wt/wt, 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, and from about 10% wt/wt to about 20% wt/wt, of the composition.

The particulates described herein can be used in various compositions such as toothpastes, foodstuffs, cosmetics, rubbers, plastics and with pharmaceuticals.

Suitable cosmetic products include, for example and are not limited to the following: baby powder, pressed powder, lipstick, soaps, creams, scrubs (exfoliating scrubs such as face washing scrubs, body washing scrubs, hand washing soaps), lotions, body gels, sun screens, facial creams, eye creams, eye shadows, make up powder, and/or for foundation make up.

Alternatively, the particulates described herein can be used as suspending agents, viscosity increasing agents, etc.

Applications in the food industry include but are limited to, for example, flow improving agents to improve flowability of powders, avoiding blockage during conveying, dosing or filling of a powdered product, such as milk powder, soybean powder, vegetable powder, yeast powder, starch or egg powder; anticaking, such that the powdered material remains freely flowing even when stored under pressure or varying ambient conditions with coffee and cocoa powders, cream powders, seasonings and yeast powders.

Further the particulates described herein can be used as drying aids. For example, the particulates described herein can be coated onto a surface to help reduce clogging of the device, such as a mill or a dryer, during grinding of a material or a drying of a material, such as milk powder, cream powder, plant extract, or seasonings, such as chili powder, pepper, etc.

The particulates described herein can serves as carriers. The particulates can be added to liquids or pastes to help convert the materials into a free flowing material which makes it easier to handle and/or store.

Additionally, the particulates described herein can be used as a replacement for titanium dioxide in confectionaries, baked goods, cheese, icings, toppings and food supplements. Generally, the particulate material is not added in excess of 1% to 2% by weight of the food. These can be included but not limited to, for example, coatings of candy, jelly, flour, vermicelli, milk, dairy products, bean-curd and soybean milk.

In terms of pharmaceuticals, the particulates described herein can be included in various coatings for medicines, such as tablets and in capsules. Further, the particulates described herein can be used as carriers or fillers for the delivery of pharmaceutical agent agents in tablet or capsule form.

The particulates described herein can be colored or “dyed”. It should be understood that the term “colored” as associated with a coloring agent is intended to encompass materials that coat the particle, embed themselves into the particle, adhere to the particle, etc. so as to effect coloration of the particle. In one aspect, the color is not removed from the particle while in use.

The colorants or coloring agent, used interchangeably, can be natural materials such as plant extracts or inorganic materials.

For example, natural colorants include, but are not limited to, curcumin extract, lutein, lutein ester, safflower red, safflower yellow, gardenia yellow, natural carotene, beta-carotene, annatto extract, paprika red, paprika extract, carminic acid extract, carmine extract, beetroot extract red, grape skin extract, black carrot extract, red cabbage extract, purple sweet potato extract, radish extract red, lycopene, chlorophyll, copper chlorophyll, sodium copper chlorophyll, spirulina blue, gardenia blue, gardenia red, elderberry extract, monascus red cocoa pigment, tomato red, orange yellow, riboflavin, black bean red, black currant red, red rice red, monascus yellow, peanut skin red, rose laevigata michx brown, coreopsis yellow, paprika orange, uguisukagura red, roselle red, basella rubra red, mulberry red, natural amaranthus red, mynica red, gromwell red, all of which are commercially available.

Exemplary non-natural colorants include, but are not limited to, β-apo-8′-carotenal, erythrosine, indigotine, caramel colour, quinolone yellow, brilliant blue, tartrazine, sunset yellow, carmoisine, amaranth, new red, carmine cochineal, ponceau 4R, allura red, lac dye red, all of which are commercially available.

It should be understood that the colorant, when combined with the particulate, can coat the particulate, can be absorbed, can be adsorbed, can penetrate into pores of the particulate, etc. The colorant becomes color fast to the particulate so that the colorant does not “bleed” or has minimal bleeding while in use under the given conditions, such as washing or scrubbing the epidermis for example.

Alternatively, the particulate material can be whitened or bleached by treating the particulate ivory nut material with an oxidant such as hydrogen peroxide. Typically, the ivory nut particulate is treated with an aqueous solution of 0.1% to about 5% (v/v) of an oxidant, such as hydrogen peroxide or sodium hypochlorite, at a temperature of from about 60° C. to about 90° C. from 1 to about 6 hours. Alternatively, an aqueous ethanol solution of hydrogen peroxide 5% (95% ethanol) can be used with the particulate ivory nut to bleach the material. Generally, the bleaching process is conducted over a period of about 6 to 10 hours at about 10 to about 60° C. With either process, the bleached or whitened ivory nut particulate material is then rinsed at least once, more particularly three times, with water to remove remaining oxidant.

The particulates described herein can be coated or surface treated. Coatings include, but are not limited to an oil, an antioxidant, a surfactant, a wax or combinations thereof.

Suitable oils include, but are not limited to, mineral oil, olive oil, white oil (paraffin oil), plant essential oil, palm oil, bilberry seed oil and mixtures thereof.

Suitable antioxidants include, but are not limited to, glutathione and enzymes (e.g., catalase and superoxide dismutase), vitamin A, vitamin C, vitamin E, green tea extract and esters thereof, grape seed extract and esters thereof, lemon balm extract, rosemary extract, frolic acid, oryzanol and mixtures thereof.

Surfactants are compounds that lower the surface tension of a liquid, allowing easier spreading upon a surface, and lowering of the interfacial tension between two liquids, or between a liquid and a solid. Surfactants may act as detergents, wetting agents, emulsifiers, foaming agents, and/or as dispersants. Surfactants are usually organic compounds that are amphiphilic, meaning they contain both hydrophobic groups (e.g., tails) and hydrophilic groups (e.g., heads). Therefore, a surfactant molecule contains both a water insoluble (or oil soluble component) and a water soluble component. For example, in water, surfactant molecules will migrate to the water surface, where the insoluble hydrophobic group may extend out of the bulk water phase, either into the air or, if water is mixed with an oil, into the oil phase, while the water soluble head group remains in the water phase. This alignment and aggregation of surfactant molecules at the surface, acts to alter the surface properties of water at the water/air or water/oil interface.

The most accepted classification of surfactants is based on their dissociation in water. Generally a surfactant, also called a surface active agent, includes types of ionic surfactant and nonionic surfactants. Ionic surfactants are classified in three general categories: anionic, cationic and zwitterionic (amphoteric) surfactants.

Anionic surfactants have a permanent anion, such as a sulfate, sulfonate and phosphate anion associated with the surfactant or has a pH-dependent anion, for example, a carboxylate.

Sulfates can be alkyl sulfate or alkyl ether sulfates.

Suitable alkyl sulfates include, but are not limited to, ammonium lauryl sulfate or sodium lauryl sulfate (SDS). Suitable alkyl ether sulfates include, but are not limited to, sodium laureth sulfate, also known as sodium lauryl ether sulfate (SLES) or sodium myreth sulfate.

Suitable sulfonates include, but are not limited to, docusate (dioctyl sodium sulfosuccinate), fluorosurfactants that are sulfonated and alkyl benzene sulfonates.

Typical sulfonated fluorosurfactants include, but are not limited to, perfluorooctanesulfonate (PFOS) or perfluorobutanesulfonate.

Phosphates are typically alkyl aryl ether phosphates or alkyl ether phosphates.

Carboxylates are typically alkyl carboxylates, such as fatty acid salts (soaps), such as for example, sodium stearate. Alternatively, the carboxylate can be, but is not limited to, sodium lauryl sarcosinate. In another alternative aspect, the carboxylate includes but is not limited to a carboxylated fluorosurfactant, such as perfluorononanoate, or perfluorooctanoate (PFOA or PFO).

In one aspect, the carboxylate can be attached to a cellulosic structure, such as in carboxymethylcellulose (CMC). Various salts and derivatives of this are available, such as the sodium and calcium salts of CMC. For example, carboxymethylcellulose or cellulose gum is a cellulose derivative with carboxymethyl groups (—CH₂—COOH) bound to some of the hydroxyl groups of the glucopyranose monomers that make up the cellulose backbone. It is often used as its sodium salt, sodium carboxymethyl cellulose.

Carboxymethylcellulose, as is well-known in the art, may have varying degrees of substitution, a “degree of substitution” referring to the number of derivatizing groups, herein carboxymethyl, per each monomer unit on the average. A particularly preferred carboxymethylcellulose has a degree of substitution of about 0.7 and a molecular weight of about 80 kD.

Cationic surfactants are dissociated in water into an amphiphilic cation and an anion, most often as a halogen. This class generally corresponds to nitrogen compounds such as fatty amine salts and quaternary ammoniums, with one or several long alkyl chains derived from natural fatty acids. These surfactants are generally more expensive than anionics, because of a the high pressure hydrogenation reaction to be carried out during their synthesis.

One kind of cationic surfactant is typically based on pH-dependent primary, secondary or tertiary amines. The primary amines become positively charged at a pH<10, and the secondary amines become charged at a pH<4. One example is octenidine dihydrochloride.

Another type of cationic surfactant is based on permanently charged quaternary ammonium cations, such as alkyltrimethylammonium salts. These include but are not limited to cetyl trimethylammonium bromide (CTAB), hexadecyl trimethyl ammonium bromide, cetyl trimethylammonium chloride (CTAC), cetylpyridinium chloride (CPC), polyethoxylated tallow amine (POEA), benzalkonium chloride (BAC), benzethonium chloride (BZT), 5-Bromo-5-nitro-1,3-dioxane, dimethyldioctadecylammonium chloride and dioctadecyldimethylammonium bromide (DODAB).

When a single surfactant molecule exhibits both anionic and cationic dissociations it is called amphoteric or zwitterionic. Zwitterionic (amphoteric) surfactant is based on primary, secondary or tertiary amines or quaternary ammonium cation also having a sulfonate, carboxylate or a phosphate.

Suitable zwitterionic surfactants include, but are not limited to, CHAPS (3-[(3-Cholamidopropyl)dimethylammonio]-1-propanesulfonate) or a sultaine. The sultaine is typically cocamidopropyl hydroxysultaine.

In one aspect, the carboxylate cation is an amino acid, imino acid or betaine. In one aspect, the betaine is typically cocamidopropyl betaine.

When the zwitterionic surfactant includes a phosphate, lecithin is often chosen as the counterion.

Nonionic surfactants are another class of surfactants. They do not ionize in aqueous solution because their hydrophilic group does not dissociate. Suitable hydrophilic groups include alcohols, phenols, ethers, esters, or amides. A large number nonionic surfactants are rendered hydrophilic by the presence of a polyethylene glycol chain obtained by the polycondensation of ethylene oxide.

Nonionic surfactants include, but are not limited to, fatty alcohols, polyoxyethylene glycol alkyl ethers (Brij), polyoxypropylene glycol alkyl ethers, glucoside alkyl ethers, polyoxyethylene glycol octylphenol ethers, polyoxyethylene glycol alkylphenol ethers, glycerol alkyl esters, polyoxyethylene glycol sorbitan alkyl esters, sorbitan alkyl esters, cocamide MEAs, cocamide DEAs, dodecyl dimethylamine oxides, block copolymers of polyethylene glycol and polypropylene glycols.

Suitable fatty alcohols include, but are not limited to, cetyl alcohol, stearyl alcohol, cetostearyl alcohol (consisting predominantly of cetyl and stearyl alcohols) and oleyl alcohol.

Suitable polyoxyethylene glycol alkyl ethers, include but are not limited to (Brij), for example CH₃—(CH₂)₁₀₋₁₆—(O—C₂H₄)₁₋₂₅—OH, or octaethylene glycol monododecyl ether or pentaethylene glycol monododecyl ether.

Suitable polyoxypropylene glycol alkyl ethers include CH₃—(CH₂)₁₀₋₁₆—(O—C₃H₆)₁₋₂₅—OH.

Suitable glucoside alkyl ethers include CH₃—(CH₂)₁₀₋₁₆—(O-Glucoside)₁₃-OH, and, for example, include decyl glucoside, lauryl glucoside, and octyl glucoside.

Suitable polyoxyethylene glycol octylphenol ethers include C₈H₁₇—(C₆H₄)—(O—C₂H₄)₁₋₂₅—OH. One exemplary material is TRITON X-100.

Suitable polyoxyethylene glycol alkylphenol ethers include C₉H₁₉—(C₆H₄)—(O—C₂H₄)₁₋₂₅—OH. One example is Nonoxynol-9.

In one aspect, a suitable glycerol alkyl ester is glyceryl laurate.

In another aspect, a suitable polyoxyethylene glycol sorbitan alkyl ester is polysorbate.

In still another aspect, suitable sorbitan alkyl esters are referred to as SPAN, e.g., SPAN-20, sorbitan monolaurate.

In yet another aspect, suitable block copolymers of polyethylene glycol and polypropylene glycol are typically referred to as poloxamers.

In a micelle, the lipophilic tails of the surfactant molecules remain on the inside of the micelle, due to unfavorable interactions. The polar “heads” of the micelles, due to favorable interactions with water, form a hydrophilic outer layer that in effect protects the hydrophobic core of the micelle. The compounds that make up a micelle are typically amphiphilic in nature, meaning that not only are micelles soluble in protic solvents, such as water, but also in aprotic solvents and can act as a reverse micelle. Surfactants reduce the surface tension of water by adsorbing at the liquid-gas interface. They also reduce the interfacial tension between oil and water by adsorbing at the liquid-liquid interface.

Many surfactants can also assemble in the bulk solution into aggregates. Examples of such aggregates are vesicles and micelles. The concentration at which surfactants begin to form micelle is known as the critical micelle concentration (CMC). When micelles form in water, their tails form a core that can encapsulate an oil droplet, and their (ionic/polar) heads form an outer shell that maintains favorable contact with water. When surfactants assemble in oil, the aggregate is referred to as a reverse micelle. In a reverse micelle, the heads are in the core and the tails maintain favorable contact with oil. Surfactants are also often classified into four primary groups; anionic, cationic, non-ionic, and zwitterionic (dual charge).

Thermodynamics of the surfactant systems are of great importance, theoretically and practically. This is because surfactant systems represent systems between ordered and disordered states of matter. Surfactant solutions may contain an ordered phase (micelles) and a disordered phase (free surfactant molecules and/or ions in the solution).

As mentioned above, nonionic surfactants, for example, a saccharide or a polysaccharide can act as a surfactant and includes edible gums.

The edible gum can be derived from microbial polysaccharides, plant saccharides, marine algal saccharides, or the mixture thereof.

For example, the microbial polysaccharides can be selected from the group of xanthan gum, gellan gum, dextran, scleroglucan, pullulan gum, curdlan and the mixture thereof.

Suitable plant saccharides include pectin, gum arabic, gum tragacanth, karaya gum, guar gum, carob gum, tara gum, konjac gum, tamarind gum, tragacanth, or the mixture thereof

Suitable marine algal polysaccharides are algin, agar, carrageenan, or the mixture thereof.

Exemplary surfactants include, but are not limited to, polyethylene glycols, polyethylene oxides, polyoxyethylene ether (stearates), phospholipids, lysophospholipids and mixtures thereof.

Suitable waxes include, but are not limited to, paraffinic waxes, beeswax, carnuba wax, lanolin wax, rice bran wax, mink wax, spent grain wax and combinations thereof.

The following paragraphs enumerated consecutively from 1 through 25 provide for various aspects of the present invention. In one embodiment, in a first paragraph (1), the present invention provides a composition comprising a particulate ivory nut or particulate hydrolyzed or extracted ivory nut (mannan) material.

2. The composition of paragraph 1, wherein the particulate material has a particle size that is granular or powdered of less than 4000 microns, more particularly 3500 microns, 3000 microns, 2500 microns or 2000 microns (μm).

3. The composition of paragraph 2, wherein the particle size is less than 1500 microns.

4. The composition of paragraph 3, wherein the particle size is less than 1000 microns.

5. The composition of paragraph 4, wherein the particle size is less than 500 microns.

6. The composition of paragraph 5, wherein the particle size is less than 200 microns.

7. The composition of paragraph 6, wherein the particle size is less than 100 microns.

8. The composition of paragraph 7, wherein the particle size is less than 50 microns.

9. The composition of paragraph 8, wherein the particle size is less than 20 microns.

10. The composition of paragraph 9, wherein the particle size is less than 10 microns.

11. The composition of paragraph 10, wherein the particle size is less than 5 microns.

12. The composition of paragraph 11, wherein the particle size is less than 1 microns.

13. The composition of paragraph 12, wherein the particle size is less than 0.2 microns.

14. The composition of any of paragraphs 1 through 13, wherein the particulate material is colored with a coloring agent.

15. The composition of paragraph 14, wherein the coloring agent is oil soluble, water soluble, or both.

16. The composition of either of paragraphs 14 and 15, wherein the coloring agent is a natural organic material or an inorganic material.

17. The composition of any of paragraphs 1 through 13, wherein the particulate is bleached.

18. The composition of paragraph 17, wherein the bleached particulate is white in color.

19. The composition of any of paragraphs 1 through 18, wherein the particulate is coated.

20. The composition of paragraph 20, wherein the coating is an oil, an antioxidant, or a wax.

21. The composition of any of paragraphs 1 through 20, further comprising a foodstuff, a toothpaste, a cosmetic composition, a rubber, a plastic, or a pharmaceutical agent.

22. The composition of paragraph 21, wherein the cosmetic composition is a baby powder, a pressed powder, lipstick, a body scrub, a soap, a lotion, a cream, a sun screen or a shampoo.

23. The composition of paragraph 21, wherein the toothpaste further comprises silicon dioxide, calcium carbonate, calcium hypophosphate, aluminum oxide, xanthan gum, sorbitol, menthol, ale powder, sodium benzoate, ethanol, holly oil, spearmint, water and combinations thereof.

24. The composition of any of paragraphs 1 through 23, wherein the particulate is spherical, elliptical, irregular in form, shards, and can be amorphous or crystalline.

25. The composition of any of paragraphs 1 through 24, wherein the particulate is used as a UV filter.

26. The composition of any of paragraphs 21 through 24, wherein the percentage of the particulate within the composition is from 0.1% by weight to 99% by weight.

27. A composition comprising a mixture of talc (e.g., micronized talc) and particulate ivory nut or talc (e.g., micronized talc) and particulate hydrolyzed ivory nut (mannan) material or talc and extracted ivory nut (mannan) material wherein the mixture can be used as the composition of any paragraph of 21-25.

28. A composition comprising a mixture of titanium dioxide and particulate ivory nut or titanium dioxide and particulate hydrolyzed ivory nut (mannan) material or extracted ivory nut (mannan) material wherein the mixture can be used as the composition of any paragraph of 21-25.

29. A composition comprising a mixture of starch or modified starch and particulate ivory nut or starch or modified starch and particulate hydrolyzed ivory nut (mannan) material or starch or modified starch and extracted ivory nut (mannan) material wherein the mixture can be used as the composition of any paragraph of 21-25.

30. The composition of any paragraph 27 through 29, wherein the percentage of the particulate ivory nut or particulate hydrolyzed ivory nut (mannan) material or extracted ivory nut (mannan) material of the composition is from 0.1% by weight to 99.9% by weight.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

### Natural Powder Products Used in Cosmetic, Toothpaste and Healthcare Products

### Example 1

Preparation Method:

Deionized water, glycerin, sorbitol, carboxymethyl cellulose, sodium lauryl sulfate was added to a reaction kettle as detailed in the formulae described below, fully agitating the mixture, and heating the mixture up to 75° C. and maintained at 75° C. for 3 hours. The materials were completely dissolved. The solution was cooled to 45° C., and the ivory nut powder was slowly added into the liquid. The mixture was homogenized within 15 minutes by use of a high pressure homogenizer. The mixture was the filtered with 300 microns (μm) sieve and tested. (wt %=weight percent)

Toothpaste Formula:

| | | |
|---|---|---|
| Ivory Nut Powder | 45 | wt % |
| Glycerin | 10 | wt % |
| Sorbitol | 10 | wt % |
| Carboxymethyl cellulose | 1 | wt % |
| Sodium lauryl sulfate | 2 | wt % |
| Deionized water | 32 | wt % |

In this formula, ivory nut powder was composed of one or more kinds of different particle sizes of ivory nut powder. It is preferred to include two kinds of ivory nut powders that are ivory nut powder A (the particle size range is 10-30 μm) and ivory nut powder B (the particle size range is 20-45 μm). The mass ratio of ivory nut powder A to ivory nut powder B is 20/80 to 80/20.

### Example 2

Preparation Method:

Deionized water, glycerin, sorbitol, mannitol, sorbitol, hydroxypropyl cellulose and sodium alkyl sulfate was added to a reaction kettle as detailed in the formulae described below, fully agitating the mixture, and heating the mixture up to 75° C. and maintained at 75° C. for 3 hours. The materials were completely dissolved. The solution was cooled to 45° C., and the ivory nut powder was slowly added into the liquid. The mixture was homogenized within 15 minutes by use of a high pressure homogenizer. The mixture was the filtered with 300 microns (μm) sieve and tested. (wt %=weight percent)

Toothpaste Formula:

| | | |
|---|---|---|
| Ivory Nut Powder | 50 | wt % |
| Glycerin | 5 | wt % |
| Mannitol | 5 | wt % |
| Sorbitol | 5 | wt % |
| hydroxypropyl cellulose | 1.2 | wt % |
| Sodium alkyl sulfate | 1.8 | wt % |
| Deionized water | 32 | wt % |

In this formula, ivory nut powder was composed of one or more kinds of different particle sizes of ivory nut powder. It is preferred to include two kinds of ivory nut powders that are ivory nut powder A (the particle size range is 10-30 μm) and ivory nut powder B (the particle size range is 20-45 μm). The mass ratio of ivory nut powder A to ivory nut powder B is 20/80 to 80/20.

### Example 3

Deionized water, glycerin, sorbitol, sorbitol, hydroxypropyl cellulose, sorbitan monostearate polyoxyethylene ether and sodium alkyl sulfate was added to a reaction kettle as detailed in the formulae described below, fully agitating the mixture, and heating the mixture up to 75° C. and maintained at 75° C. for 3 hours. The materials were completely dissolved. The solution was cooled to 45° C., and the ivory nut powder was slowly added into the liquid. The mixture was homogenized within 15 minutes by use of a high pressure homogenizer. The mixture was the filtered with 300 microns (μm) sieve and tested. (wt %=weight percent)

Toothpaste Formula:

| | | |
|---|---|---|
| Ivory Nut Powder | 55 | wt % |
| Glycerin | 5 | wt % |
| Sorbitol | 5 | wt % |
| hydroxypropyl cellulose | 1 | wt % |
| Sorbitan monostearate polyoxyethylene ether | 0.5 | wt % |
| sodium lauryl sulfate | 1.5 | wt % |
| Deionized water | 32 | wt % |

In this formula, ivory nut powder was composed of one or more kinds of different particle sizes of ivory nut powder. It is preferred to include two kinds of ivory nut powders that are ivory nut powder A (the particle size range is 10-30 μm) and ivory nut powder B (the particle size range is 20-45 μm). The mass ratio of ivory nut powder A to ivory nut powder B is 20/80 to 80/20.

### Example 4

Preparation Method:

A mixture of ivory nut powder and mannan, boric acid, zinc stearate, magnesium carbonate was added to a universal mixer to blend. The mixture was filtered through a 300 micron (μm) sieve, to which was added plant essential oil, followed by mixing in the universal mixer for 2 hours. The mixture was filtered through a 300 μm sieve and tested. (wt %=weight percent)

Baby Powder Formula:

| | | |
|---|---|---|
| Mixture of Ivory Nut Powder and mannan | 88 | wt % |
| Boric acid | 4 | wt % |
| Zinc stearate | 1.5 | wt % |
| Magnesium carbonate | 6.0 | wt % |
| Plant essential oil | 0.55 | wt % |

The mass ratio of ivory nut powder to mannan in the mixture was 1/99 to 99/1. The particle size of ivory nut powder was 90%≤40 μm and 50%≤25 μm. The particle size of mannan was 90%≤5.5 μm and 50%≤2.2 μm.

### Example 5

Preparation Method:

A mixture of ivory nut powder and mannan, boric acid, zinc stearate, magnesium carbonate was added to a universal mixer to blend. The mixture was filtered through a 300 micron (μm) sieve, to which was added plant essential oil, followed by mixing in the universal mixer for 2 hours. The mixture was filtered through a 300 μm sieve and tested. (wt %=weight percent)

Baby Powder Formula:

| | | |
|---|---|---|
| Ivory Nut Powder and mannan | 86 | wt % |
| Boric acid | 4 | wt % |
| Zinc stearate | 3 | wt % |
| Magnesium carbonate | 6 | wt % |
| Plant essential oil | 1 | wt % |

The mass ratio of ivory nut powder to mannan in the mixture was 1/99 to 99/1. The particle size of ivory nut powder was 90%≤40 μm and 50%≤25 μm. The particle size of mannan was 90%≤5.5 μm and 50%≤2.2 μm.

### Example 6

Preparation Method

A mixture of ivory nut powder and mannan, boric acid, zinc stearate, magnesium carbonate was added to a universal mixer to blend. The mixture was filtered through a 300 micron (μm) sieve, to which was added plant essential oil, followed by mixing in the universal mixer for 2 hours. The mixture was filtered through a 300 μm sieve and tested. (wt %=weight percent)

Baby Powder Formula:

| | | |
|---|---|---|
| Base powder | 89 | wt % |
| Boric acid | 3.5 | wt % |
| Zinc stearate | 1.0 | wt % |
| Magnesium carbonate | 6 | wt % |
| Plant essential oil | 0.5 | wt % |

The mass ratio of ivory nut powder to mannan in the mixture was 1/99 to 99/1. The particle size of ivory nut powder was 90%≤40 μm and 50% μm. The particle size of mannan was 90%≤5.5 μm and 50%≤2.2 μm.

### Example 7

Preparation Method

White oil, Vaseline, almond oil, paraffin, sorbitan stearate and sorbitan monostearate polyoxyethylene ether was added to a reaction kettle with agitation and heated to 75° C. A mixture of ivory nut powder and mannan was slowly added to the mixture and homogenized over a period of about 15 minutes with a high pressure homogenizer. The mixture was then cooled to 40° C., filtered through a 300 micron (μm) sieve and tested. (wt %=weight percent)

Foundation Formula:

| | | |
|---|---|---|
| Ivory Nut Powder and mannan | 30 | wt % |
| White oil | 30 | wt % |
| Vaseline | 15 | wt % |
| Almond oil | 10 | wt % |
| Paraffin | 11 | wt % |
| Sorbitan stearate | 2 | wt % |
| Sorbitan monostearate polyoxyethylene ether | 2 | wt % |

The mass ratio of ivory nut powder to mannan in the mixture was 1/99 to 99/1. The particle size of ivory nut powder was 90%≤20 μm and 50%≤8 μm. The particle size of mannan was 90%≤4 μm and 50%≤1 μm.

### Example 8

Preparation Method

White oil, Vaseline, palm kernel oil, paraffin, glyceryl monostearate, sucrose stearate and sorbitan monostearate polyoxyethylene ether was added to a reaction kettle with agitation and heated to 75° C. An ivory nut powder and mannan mixture was slowly added to the mixture and homogenized over a period of about 15 minutes with a high pressure homogenizer. The mixture was then cooled to 40° C., filtered through a 300 micron (μm) sieve and tested. (wt %=weight percent)

Foundation Formula:

| | | |
|---|---|---|
| Ivory Nut Powder and mannan | 35 | wt % |
| White oil | 20 | wt % |
| Vaseline | 25 | wt % |
| Palm kernel oil | 10 | wt % |
| Paraffin | 6 | wt % |
| Glyceryl monostearate | 0.5 | wt % |
| Sucrose stearate | 1.5 | wt % |
| Sorbitan monostearate polyoxyethylene ether | 2 | wt % |

The mass ratio of ivory nut powder to mannan in the mixture was 1/99 to 99/1. The particle size of ivory nut powder was 90%≤20 m and 50%≤8 μm. The particle size of mannan was 90%≤4 μm and 50%≤1 μm.

### Example 9

Preparation Method:

White oil, Vaseline, palm kernel oil, paraffin, sorbitan stearate and sorbitan monostearate polyoxyethylene ether was added to a reaction kettle with agitation and heated to 75° C. A mixture of ivory nut powder and mannan was slowly added to the mixture and homogenized over a period of about 15 minutes with a high pressure homogenizer. The mixture was then cooled to 40° C., filtered through a 300 micron (μm) sieve and tested. (wt %=weight percent)

Foundation Formula:

| | | |
|---|---|---|
| Ivory Nut Powder and mannan | 33 | wt % |
| White oil | 27 | wt % |
| Vaseline | 15 | wt % |
| Palm kernel oil | 12 | wt % |
| Paraffin | 8 | wt % |
| Sorbitan stearate | 3 | wt % |
| Sorbitan monostearate polyoxyethylene ether | 2 | wt % |

The mass ratio of ivory nut powder to mannan in the mixture was 1/99 to 99/1. The particle size of ivory nut powder was 90%≤20 μm and 50%≤8 μm. The particle size of mannan was 90%≤4 μm and 50%≤1 μm.

### Example 10

Preparation Method:

Olive oil, glycerin, ascorbate monophosphate, glyceryl monostearate and deionized water was added to a reaction kettle with agitation and heated to 75° C. A mixture of ivory nut powder and mannan was slowly added to the mixture and homogenized over a period of about 15 minutes with a high pressure homogenizer. The mixture was then cooled to 40° C., filtered through a 300 micron (μm) sieve and tested. (wt %=weight percent)

Sunscreen Cream Formula

| | | |
|---|---|---|
| Ivory Nut Powder and mannan | 12 | wt % |
| Olive oil | 15 | wt % |
| glycerin | 5 | wt % |
| ascorbate monophosphate | 1 | wt % |
| glyceryl monostearate | 5 | wt % |
| deionized water | 62 | wt % |

The mass ratio of ivory nut powder to mannan in the mixture was 1/99 to 99/1. The particle size of ivory nut powder was 90%≤40 μm and 50%≤25 μm. The particle size of mannan was 90%≤5.5 μm and 50%≤2.2 μm.

### Example 11

Preparation Method:

Almond oil, sorbitol, ascorbyl palmitate, sucrose stearate and deionized water was added to a reaction kettle with agitation and heated to 75° C. A mixture of ivory nut powder and manna was slowly added to the mixture and homogenized over a period of about 15 minutes with a high pressure homogenizer. The mixture was then cooled to 40° C., filtered through a 300 micron (μm) sieve and tested. (wt %=weight percent)

Sunscreen Cream Formula:

| | |
|---|---|
| Ivory Nut Powder and mannan | 15 |
| Almond oil | 13 |
| Sorbitol | 5 |
| Ascorbyl palmitate | 1 |
| Sucrose stearate | 6 |
| Deionized water | 60 |

The mass ratio of ivory nut powder to mannan in the mixture was 1/99 to 99/1. The particle size of ivory nut powder was 90%≤30 μm and 50%≤10 μm. The particle size of mannan was 90%≤5 μm and 50%≤2 μm.

### Example 12

Preparation Method:

Palm kernel oil, glycerin, licorice extract, glyceryl monostearate and deionized water was added to a reaction kettle with agitation and heated to 75° C. A mixture of ivory nut powder and mannan was slowly added to the mixture and homogenized over a period of about 15 minutes with a high pressure homogenizer. The mixture was then cooled to 40° C., filtered through a 300 micron (μm) sieve and tested. (wt %=weight percent)

Sunscreen Cream Formula:

| | |
|---|---|
| Ivory Nut Powder and mannan | 10 |
| Palm kernel oil | 11 |
| Glycerin | 6 |
| Licorice extract | 1 |
| Glyceryl monostearate | 5 |
| Deionized water | 67 |

The mass ratio of ivory nut powder to mannan in the mixture was 1/99 to 99/1. The particle size of ivory nut powder was 90%≤30 μm and 50%≤10 μm. The particle size of mannan was 90%≤5 μm and 50%≤2 μm.

The Preparation of dyed Ivory nut powder products:

### Example 1

Raw Materials:

| Raw material name | Specification | Quantity | |
|---|---|---|---|
| Bilberry extract | UV Anthocyanin content ≥25% | 2 | g |
| Ivory nut powder | Through 80 mesh sieve | 30 | g |
| | Not through 100 mesh sieve | | |
| Deionzed water | None | 200 | ml |

Preparation Method:

2 g Bilberry extract (Ningbo Green-Health Pharmaceutical Co., Ltd.) and 200 ml deionized water were added to a reaction kettle with agitation until the bilberry extract was dissolved completely at room temperature. To the solution was slowly added 30 g ivory nut powder (particle size as indicated above) and the resulting mixture was stirred for 24 hours at room temperature. The mixture was filtered to obtain a purple solid powder (30 g). The purple particulate was ground with a universal grinder, dried at 80° C., and sieved with a 80 mesh sieve to provide a final purple powder suitable for use.

### Example 2

Raw Materials:

| Raw material name | Specification | Quantity | |
|---|---|---|---|
| Bilberry extract | UV Anthocyanin content ≥25% | 10 | g |
| Ivory nut powder | Through 80 mesh sieve | 30 | g |
| | Not through 100 mesh sieve | | |
| Deionzed water | None | 200 | ml |

Preparation Method:

10 g Bilberry extract (Ningbo Green-Health Pharmaceutical Co., Ltd.) and 200 ml deionized water were added to a reaction kettle with agitation until the bilberry extract was dissolved completely at room temperature. To the solution was slowly added 30 g ivory nut powder (particle size as indicated above) and the resulting mixture was stirred for 24 hours at room temperature. The mixture was filtered to obtain a purple solid powder (27.3 g). The purple particulate was ground with a universal grinder, dried at 80° C., and sieved with a 80 mesh sieve to provide a final purple powder suitable for use.

### Example 3

Raw Materials:

| Raw material name | Specification | Quantity | |
|---|---|---|---|
| Raddish red | Color value ≥80 | 2 | g |
| Ivory nut powder | Through 80 mesh sieve | 30 | g |
| | Not through 100 mesh sieve | | |
| Deionzed water | None | 200 | ml |

Preparation Method:

2 g radish red (Wuhan Green Food Biological Engineering Co., Ltd.) and 200 ml deionized water were added to a reaction kettle with agitation until the radish red was dissolved completely at room temperature. To the solution was slowly added 30 g ivory nut powder (particle size as indicated above) and the resulting mixture was stirred for 24 hours at room temperature. The mixture was filtered to obtain a rose red powder (30 g). The red rose particulate was ground with a universal grinder, dried at 80° C., and sieved with a 80 mesh sieve to provide a final rose red powder suitable for use

### Example 4

Raw Materials:

| Raw material name | Specification | Quantity | |
|---|---|---|---|
| Purple grape red | Color Value ≥20% | 10 | g |
| Ivory nut powder | Through 80 mesh sieve | 30 | g |
| | Not through 100 mesh sieve | | |
| Deionzed water | None | 100 | ml |

Preparation Method:

10 g purple grape red (E. & J. Gallo Winery) and 100 ml deionized water were added to a reaction kettle with agitation until the purple grape red was dissolved completely at room temperature. To the solution was slowly added 30 g ivory nut powder (particles pass through 80 mesh but not through 100 mesh; 150 microns to 200 microns) and the resulting mixture was stirred for 24 hours at room temperature. The mixture was filtered to obtain a purple red powder (27.9 g). The purple red particulate was ground with a universal grinder, dried at 60° C., and sieved with a 80 mesh sieve to provide a final purple red powder suitable for use.

### Example 5

Raw Materials:

| Raw material name | Specification | Quantity | |
|---|---|---|---|
| Gardenia yellow | Color value ≥200 | 5 | g |
| Ivory nut powder | Through 80 mesh sieve | 30 | g |
| | Not through 100 mesh sieve | | |
| Deionzed water | None | 100 | ml |

Preparation Method:

5 g gardenia yellow (Wuhan Green Food Biological Engineering Co., Ltd.) and 100 ml deionized water were added to a reaction kettle with agitation until the gardenia yellow was dissolved completely at room temperature. To the solution was slowly added 30 g ivory nut powder (particles pass through 80 mesh but not through 100 mesh; 150 microns to 200 microns) and the resulting mixture was stirred for 24 hours at room temperature. The mixture was filtered to obtain a light yellow powder (26.2 g). The light yellow particulate was ground with a universal grinder, dried at 80° C., and sieved with a 80 mesh sieve to provide a final light yellow powder suitable for use.

### Example 6

Raw Materials:

| Raw material name | Specification | Quantity | |
|---|---|---|---|
| Copper chlorophyll | Content ≥17% | 10 | g |
| Ivory nut powder | Through 80 mesh sieve | 100 | g |
| | Not through 100 mesh sieve | | |
| Ethyl acetate | AR | 150 | ml |

Preparation Method:

10 g copper chlorophyll (Shandong Guangtongbao Pharmaceuticals Co., Ltd), and 150 ml ethyl acetate were added to a reaction kettle with agitation until the copper chlorophyll was dissolved completely at room temperature. To the solution was slowly added 100 g ivory nut powder (particles pass through 80 mesh but not through 100 mesh; 150 microns to 200 microns) and the resulting mixture was stirred for 24 hours at room temperature. The mixture was filtered to obtain a dark green powder (99.3 g). The dark green particulate was ground with a universal grinder, dried at 80° C., and sieved with a 80 mesh sieve to provide a final dark green powder suitable for use.

### Example 7

Raw Materials:

| Raw material name | Specification | Quantity | |
|---|---|---|---|
| Lutein ester | UV Content ≥40% | 5 | g |
| Ivory nut powder | Through 80 mesh sieve | 100 | g |
| | Not through 100 mesh sieve | | |
| Ethyl acetate | AR | 150 | ml |

5 g lutein ester (Ningbo Green-Health Pharmaceutical Co., Ltd.) and 150 ml ethyl acetate were added to a reaction kettle with agitation until the copper chlorophyll was dissolved completely at 60° C. To the solution was slowly added 100 g ivory nut powder (particles pass through 80 mesh but not through 100 mesh; 150 microns to 200 microns) and the resulting mixture was stirred for 24 hours at 60° C. The mixture was filtered to obtain a dark orange powder (100 g). The dark orange particulate was ground with a universal grinder, dried at 60° C., and sieved with a 80 mesh sieve to provide a final dark orange powder suitable for use.

### Example 8

Raw Materials:

| Raw material name | Specification | Quantity | |
|---|---|---|---|
| Paprika red | Color value ≥100 | 10 | g |
| Ivory nut powder | Through 80 mesh sieve | 100 | g |
| | Not through 100 mesh sieve | | |
| Ethyl acetate | AR | 150 | ml |

5 g paprika red (Henan Zhongda Biological Engineering Co.) and 150 ml ethyl acetate were added to a reaction kettle with agitation until the copper chlorophyll was dissolved completely at room temperature. To the solution was slowly added 100 g ivory nut powder (particles pass through 80 mesh but not through 100 mesh; 150 microns to 200 microns) and the resulting mixture was stirred for 24 hours at room temperature. The mixture was filtered to obtain a dark red powder (100 g). The dark red particulate was ground with a universal grinder, dried at 60° C., and sieved with a 80 mesh sieve to provide a final dark red powder suitable for use.

Conclusion: Ivory nut particles could be dyed by both water soluble and fat soluble colorants.

### Example 9

Raw Materials:

| Raw material name | Specification | Quantity | |
|---|---|---|---|
| Gardenia red | Color value ≥30 | 30 | g |
| Ivory nut powder | Through 80 mesh sieve | 100 | g |
| | Not through 100 mesh sieve | | |
| Deionzed water | None | 150 | ml |

30 g gardenia red (Wuhan Green Food Biological Engineering Co., Ltd.) and 150 ml deionized water were added to a reaction kettle with agitation until the gardenia red was dissolved completely at room temperature. To the solution was slowly added 30 g ivory nut powder (particles pass through 80 mesh but not through 100 mesh, 150 microns to 200 microns) and the resulting mixture was stirred for 24 hours at room temperature. The mixture was filtered to obtain a light yellow powder (36.2 g). The red particulate was ground with a universal grinder, dried at 80° C., and sieved with an 80 mesh sieve to provide a final light yellow powder suitable for use.

### EXAMPLES: THE APPLICATION OF DYED IVORY NUT POWDER PRODUCTS

### Example 1: Massage Scrub Formula

Preparation Method:

Glycerol and B phase components were added to a reaction kettle with heating and agitation until the B phase was completely dissolved at 80° C. The mixture was cooled to 45° C. and the C phase was added with agitation for about 10 minutes until the mixture was uniform. The remainder of the A phase was added to the mixture with agitation along with the D phase until the mixture was uniform. The mixture was cooled down to room temperature, tested, and collected.

Body Massage Scrub Formula:

| | |
|---|---|
| Ingredient name | Quantity/100 g |

A phase

| | | |
|---|---|---|
| Glycerol | 26.00 | g |
| Silicon dioxide | 3.00 | g |
| Kaolin | 2.00 | g |
| Ivory nut powder dyed by Paprika red | 5.00 | g |
| Deionzed water | 56.05 | g |

B phase

| | | |
|---|---|---|
| Octadecanol | 4.00 | g |

| | |
|---|---|
| Tween 20 | 2.00 |

C phase

| | | |
|---|---|---|
| Sodium lauryl sulfate | 0.80 | g |
| Deionzed water | 3.00 | g |

D phase

| | | |
|---|---|---|
| Strawberry essence | 0.15 | g |

### Example 2: Nourishing Scrub Mask Formula

Preparation Method:

Allantoin, the preservative and deionized water (B phase) were added to a main reaction kettle with agitation and heated at 85° C. until materials were completely dissolved. To a second reaction kettle was added veegum, xanthan gum, kaolin and glycerol (C phase) with agitation until the materials were completely dissolved at ambient temperature. The A phase and C phase was added to the main reaction kettle with agitation and heated to 85° C. until the mixture was dispersed uniformly. The mixture was cooled to 45° C. and the D and E phases were added with agitation over about a 10 minutes period until the mixture was uniform. The mixture was cooled down to room temperature, tested, and collected.

Nourishing Scrub Mask Formula:

| | |
|---|---|
| Ingredient name | Quantity/100 g |

A phase

| | | |
|---|---|---|
| Cetearyl alcohol | 2.00 | g |
| Cetearyl glucoside | 3.00 | g |
| hexadecyl alcohol/octadecyl alcohol | 3.00 | g |
| 2,3-Dihydroxypropyl Octadecanoate | 3.00 | g |
| White oil | 8.00 | g |
| Preservative | 0.30 | g |

B phase

| | | |
|---|---|---|
| Allantoin | 0.30 | g |
| Pheoxetol/methylparaben/ethylparaben/Nipasol | 0.30 | g |
| and butylparaben (preservative mixture) | | |
| Deionzed water | 65.91 | g |

C phase

| | | |
|---|---|---|
| Veegum | 1.00 | g |
| Xanthan gum | 0.20 | g |
| Kaolin | 3.00 | g |
| Glycerol | 9.80 | g |

D phase

| | | |
|---|---|---|
| Ivory nut powder dyed by Copper chlorophyll | 0.15 | g |

E phase

| | | |
|---|---|---|
| Green Grass Essence | 0.04 | g |

### Example 3: Deep Exfoliating Scrub Formula

Preparation Method:

The A phase was added to a reaction kettle with agitation and heated until the mixture was completely dissolved at 85° C. The B phase was added to a second reaction kettle with agitation until the mixture was completely dissolved at room temperature. The C phase was added to a third reaction kettle with agitation and heating until the mixture was uniformly dispersed at 80° C. The A phase was transferred to a main reaction kettle after filtration. The B phase was transferred to the main reaction kettle with agitation over a period of 10 minutes (agitation approximately 50 revolutions per minute “rpm”). The mixture was stirred until homogenous after about 25 minutes at 5000 rpm. The mixture was homogenized after about 5 minutes at 5000 rpm. The pH value was then determined. The mixture was cooled to 50° C. to which phase D was added with agitation over a period of 5 minutes and homogenized at 2000 rpm. The mixture was cooled down to 40° C. at which time the mixture became a paste and was stirred for about 3 minutes at 15 to 20 rpm. The mixture/paste was further cooled to 38° C. for sampling and then use.

Deep Exfoliating Cleansing Scrub Formula:

| | |
|---|---|
| Ingredient name | Quantity/100 g |

A phase

| lauric acid | 5.00 | g |
|---|---|---|
| tetradecanoic acid | 4.00 | g |
| hexadecanoic acid | 12.00 | g |
| Octadecanoic acid | 8.00 | g |
| Pearlescing agent | 2.00 | g |
| 2,3-Dihydroxypropyl Octadecanoate | 3.00 | g |
| Castoryl Maleate | 0.50 | g |
| Methylparaben | 0.15 | g |
| Nipasol (a broad spectrum antimicrobial agent) | 0.20 | |
| Sodium lauryl ether sulfate | 8.00 | g |
| Lauramido propylamine oxide | 3.00 | g |
| Propylene glycol | 5.00 | g |
| Glycerol | 5.00 | g |

B phase

| | | |
|---|---|---|
| Glycerol | 8.00 | g |
| Ivory nut powder dyed by Lutein ester | 5.00 | g |

C phase

| | | |
|---|---|---|
| Potassium hydroxide | 5.90 | g |
| Deionzed water | 25.05 | g |

D phase

| | | |
|---|---|---|
| Orange essence | 0.20 | g |

The physical properties of examples of mannan from ivory nut:

1. Water and Oil Absorption Test

Material:

| Material name | Specification | Quantity |
|---|---|---|
| Mannan | D95 ≤ 10 μm, D50 ≤ 5 μm | 50-100 g |
| Ivory nut powder | Through 200 mesh sieve | 50-100 g |
| Tapioca starch | Cosmetic grade | 50-100 g |

The Ivory nut powder was prepared as described below as “Preparation method of Ivory Nut Powder (200 mesh)”

Mannan was provided by EPC Nature products and tapioca starch was purchased from AkzoNobel.

Bulk Density:

| Material name | Bulk density g/cm³ |
|---|---|
| Mannan | 0.2678 |
| Ivory nut powder | 0.3390 |
| Cosmetic tapioca starch | 0.4786 |

Water Absorption Test:

A 50 gram sample was weighed to which was added enough water to immerse the solid. The mixture was allowed to equilibrate for 2 hours at a temperature range of from about 20° C. to about 25° C. followed by filtration. The mass was measured along with the volume of the sample and the bulk density calculated.

Oil Absorption Test:

A 3 gram sample was weighed to which was added soybean oil in a dropwise fashion with mixing until the powder became dough like and no residual oil remained. The amount of oil absorbed was noted and then the oil absorption was calculated.

| | Volume change | | |
|---|---|---|---|
| | after immersion | Water absorption | Oil absorption |
| Material name | in water cm³/g | g/g | g/g |
| Mannan | +1.764 | 1.716 | 1.567 |
| Ivory nut powder | +1.550 | 0.692 | 0.545 |
| Cosmetic tapioca | −0.646 | 0.602 | 0.600 |
| starch | | | |

Moisture absorption and repose angle test was performed by using AST C1444-00, for measuring the angle of repose of free-flowing mold powders.

Material:

| Material name | Bulk density g/cm³ | Repose Angle ° | Quantity |
|---|---|---|---|
| Mannan | 0.2678 | 39.00 | 10-15 g |
| Micronized Talc | 0.2874 | 46.11 | 10-15 g |
| Cosmetic tapioca | 0.5024 | 45.53 | 10-15 g |
| starch | | | |

Micronized talc was purchased from Mondo Minerals.

Moisture Absorption Test:

10 g to 15 g sample were placed in temperature and humidity controlled incubator 25° C. and 95% RH (relative humidity) for 2 hours. Weight measurements were taken at 0 minutes, 20 minutes, 60 minutes and 120 minutes.

Results of Moisture Absorption Test:

| | | | | | | Quantity |
|---|---|---|---|---|---|---|
| | | | | | | change |
| Material | Quantity | 0 | 20 | 60 | 120 | per gram |
| name | g | min | min | min | min | g/100 g |
| Mannan | 12.2040 | 0 g | 0.2772 g | 0.5832 g | 0.8308 g | 6.8076 |
| Micronized | 12.3110 | 0 g | 0.0329 g | 0.0561 g | 0.0522 g | 0.4240 |
| Talc | | | | | | |
| Tapioca | 12.3436 | 0 g | 0.2495 | 0.5242 g | 0.7265 g | 5.8856 |
| starch | | | | | | |

The results showed that mannan has the best capability of moisture absorption. The micronized talc water absorption capacity was very poor.

Results of Repose Angles:

| | Repose angle | Repose angle | |
|---|---|---|---|
| | Before moisture | After moisture | Repose angle |
| Material name | absorption | absorption | change |
| Mannan | 39.00° | 35.12° | −3.88° |
| Micronized Talc | 46.11° | 46.99° | +0.88° |
| Tapioca starch | 45.53° | 49.58° | +4.05° |

The tapioca starch became sticky after moisture absorption, so the repose angle increased 4.05°. This means that the flow property of tapioca starch deteriorated after water absorption. In contrast to tapioca starch, mannan had improved flow characteristics after moisture absorption. Measurements were taken using the fixed funnel method.

Evaluation of Oil Absorption Function of Different Proportions of Mixtures that Mannan Blend with Talc, Corn Starch and Novelose

| Nr | Mannan (%) | Talc (%) | amount sunflower oil per 1 g |
|---|---|---|---|
| 1 | 0 | 100 | 1.02 |
| 2 | 10 | 90 | 1.02 |
| 3 | 25 | 75 | 1.02 |
| 4 | 50 | 50 | 1.03 |
| 5 | 75 | 25 | 1.03 |
| 6 | 90 | 10 | 1.03 |
| 7 | 100 | 0 | 1.02 |

| Nr | Mannan (%) | Corn Starch (%) | amount sunflower oil per 1 g |
|---|---|---|---|
| 1 | 100 | 0 | 1.02 |
| 2 | 90 | 10 | 1.02 |
| 3 | 75 | 25 | 1.03 |
| 4 | 50 | 50 | 1.02 |
| 5 | 25 | 75 | 1.01 |
| 6 | 10 | 90 | 1.01 |

| Nr | Mannan (%) | Novelose (%) | amount sunflower oil per 1 g |
|---|---|---|---|
| 1 | 100 | 0 | 1.02 |
| 2 | 90 | 10 | 1.02 |
| 3 | 75 | 25 | 1.02 |
| 4 | 50 | 50 | 1.02 |
| 5 | 25 | 75 | 1.02 |
| 6 | 10 | 90 | 1.02 |

| Products | Supplier |
|---|---|
| Talc | Mondo Minerals |
| Mannan | EPC Nature products |
| Novelose | National Starch & Chemicals Co. |
| Corn starch | AkzoNobel |

Bulk density of each material was approximately 0.1 g/cm³.

Oil absorption value of Mannan and Talc is almost identical. Therefore it is possible to blend Mannan with Talc without loss of quality (regarding oil absorption).

Oil absorption value of Mannan and Starch is almost identical. Therefore it is possible to blend Mannan with Starch without loss of quality (regarding oil absorption). There is no visible difference between common corn starch and high amylose corn starch (Novelose).

Covering Power Test

Material:

| Product | Batch no. | Producer |
|---|---|---|
| Titanium dioxide | 0970 | SHANGHAI JIANGHU TITANIU |
| | | WHITE PRODUCT CO., LTD. |
| Nano titanium dioxide | 0970(N) | SHANGHAI JIANGHU TITANIU |
| | | WHITE PRODUCT CO., LTD. |
| Mannan | 20170604 | EPC Nature product |
| DRY-FLO PURE | 990803C | AkzoNobel |
| Micronized Talc | 20170604 | Mondo Minerals |

Equipment:

A rectangular glass panel with black and white grids was prepared. The size of the glass panel was 100×250 mm and the size of grid on it was 25×25 mm. Grid area on the glass panel was 200×100 mm. Therefore, there are 16 white and black grids on the glass panel.

Methods:

1. The powder was dispersed into the modest volume of water. The panel was tared.

2. The turbidimetric solution was smeared on the panel by a brush until the black and white grids cannot be distinguished.

3. The panel was dried in oven with 90° C. The weight of powder in panel was obtained (W).

4. The covering power was calculated by the formula X=W/S×10000.

Results:

| Samples | Covering power(g/m²) |
|---|---|
| Titanium dioxide | 5 |
| Nano Titanium dioxide | 55 |
| Mannan | 150 |
| DRY-FLO PURE | 255 |
| Micronized Talc | 300 |

The covering powder of titanium dioxide is better than others. But in other materials, mannan has better performance than modified starch (DRY-FLO PURE) and micronized talc. This means mannan can be substituted for the other materials. The titanium dioxide had a particle size of approximately 0.1-20 microns and the particle size of the nanotitanium dioxide was approximately 0.01-0.1 microns.

Adhesiveness Test

Material:

| Products | Batchno. | Producer |
|---|---|---|
| Titanium Dioxide | 0970 | SHANGHAI JIANGHU TITANIU |
| | | WHITE PRODUCT CO., LTD. |
| Mannan | 20170604 | EPC Nature product |
| DRY-FLO PURE | 990803C | AkzoNobel |
| Tapioca starch | Cosmetic grade | AkzoNobel |
| Micronized Talc | 20170604 | Mondo Minerals |

Methods:

1. A specific area, which was 40 cm² was selected on forearm skin. Prior to treatment, the area was cleansed and dried with a hair dryer.

2. A defined weight (W1) of powder was applied to the area by hand with latex gloves.

3. Excess material was removed from the forearm by 3 vigorous shakes and collected to provide W2.

4. The weight of powder adhered in the area was obtained (W1−W2).

| Products | The quality adhered to the 40 cm² skin |
|---|---|
| Micronized Talc | 0.060 g |
| Tapioca starch | 0.040 g |
| Titanium Dioxide | 0.050 g |
| DRY-FLO PURE | 0.100 g |
| Mannan | 0.085 g |

The results showed that The modified starch (DRY-FLO PURE) was the best for adhesiveness. Mannan was much better than Titanium dioxide, Tapioca starch and micronized talc in terms of adhesiveness. This result shows that mannan is the easier for application and remains on the skin longest.

Ultraviolet Transmittance Test

Material:

| Product | Batch no. | Producer |
|---|---|---|
| Titanium dioxide | 0970 | SHANGHAI JIANGHU TITANIU |
| | | WHITE PRODUCT CO., LTD. |
| Mannan | 20170604 | EPC Nature product |
| DRY-FLO PURE | 990803C | AkzoNobel |
| Micronized Talc | 20170604 | Mondo Minerals |

Methods:

1. The powder was dispersed in water to prepare a turbidimetric solution. Titanium dioxide content ratio 0.2760% w/w, DRY-FLO PURE content ratio 5.22000% w/w, Mannan content ratio 3.2680% w/w, micronized talc content ratio 4.4820% w/w.

2. A half volume of turbidimetric solution was transferred into a new glass vessel, and then water was added until original volume.

3. Repeat step 2 four to six times, 5-7 different concentrations of turbidimetric solutions were obtained.

4. An injector was used to transfer turbidimetric solutions into 0.5 mm cuvettes. The Wavelength scan was from 190 to 550 nm.

5. The ultraviolet absorption data were sampled in wave length of 200 300 and 400 nm.

Results:

| | 400 nm | 300 nm | 200 nm |
|---|---|---|---|
| | transmittance | transmittance | transmittance |
| Titanium dioxide content | | | |
| ratio w/w | | | |
| 0.2760% | 1.17% | 1.24% | 2.25% |
| 0.1380% | 10.81% | 13.24% | 17.38% |
| 0.0690% | 32.81% | 36.31% | 42.76% |
| 0.0345% | 58.21% | 61.38% | 66.37% |
| 0.0200% | 75.34% | 76.91% | 82.04% |
| 0.0100% | 86.50% | 87.30% | 86.90% |
| 0.0050% | 92.90% | 93.11% | 91.24% |
| 0.0025% | 96.16% | 95.94% | 96.61% |
| 0.0013% | 98.63% | 98.40% | 98.17% |
| 0.0006% | 99.31% | 99.54% | 98.40% |
| DRY-FLO PURE content | | | |
| ratio w/w | | | |
| 5.2200% | 2.69% | 2.45% | 1.31% |
| 2.6100% | 16.29% | 17.14% | 11.12% |
| 1.3050% | 50.58% | 51.40% | 33.50% |
| 0.6525% | 69.82% | 69.02% | 51.52% |
| 0.3263% | 77.98% | 77.27% | 61.94% |
| 0.1631% | 89.33% | 88.72% | 76.56% |
| Mannan content | | | |
| ratio w/w | | | |
| 3.2680% | 0.34% | 0.16% | 0.01% |
| 1.6340% | 0.80% | 0.43% | 0.03% |
| 0.8170% | 6.18% | 5.58% | 5.33% |
| 0.6000% | 19.72% | 19.63% | 22.65% |
| 0.3000% | 46.56% | 46.03% | 49.77% |
| 0.1500% | 67.92% | 67.30% | 69.02% |
| 0.0750% | 79.98% | 79.80% | 81.10% |
| 0.0375% | 89.54% | 89.74% | 92.26% |
| 0.0188% | 92.47% | 94.19% | 96.83% |
| 0.0094% | 96.16% | 96.16% | 96.38% |
| Micronized Talc | | | |
| w/w | | | |
| 4.4820% | 0.38% | 0.14% | 0.01% |
| 2.2410% | 1.48% | 1.21% | 0.57% |
| 1.1205% | 6.46% | 5.28% | 3.80% |
| 0.6000% | 20.42% | 18.66% | 16.41% |
| 0.3000% | 45.29% | 42.76% | 39.45% |
| 0.1500% | 66.37% | 64.12% | 59.43% |
| 0.0750% | 80.17% | 77.98% | 73.62% |
| 0.0375% | 88.92% | 87.70% | 82.60% |
| 0.0188% | 93.11% | 91.83% | 89.74% |

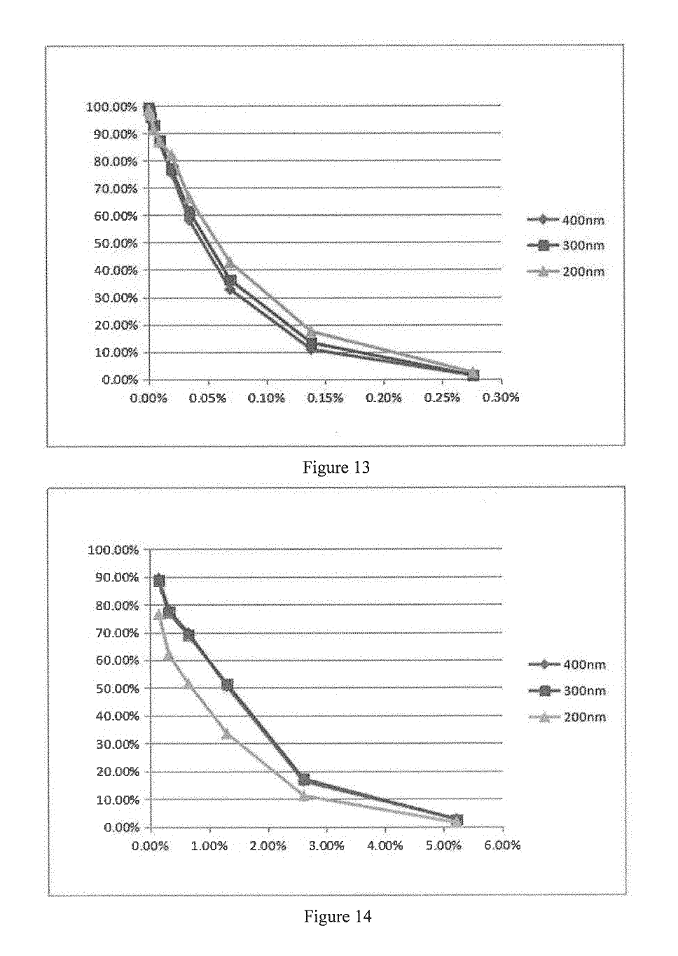

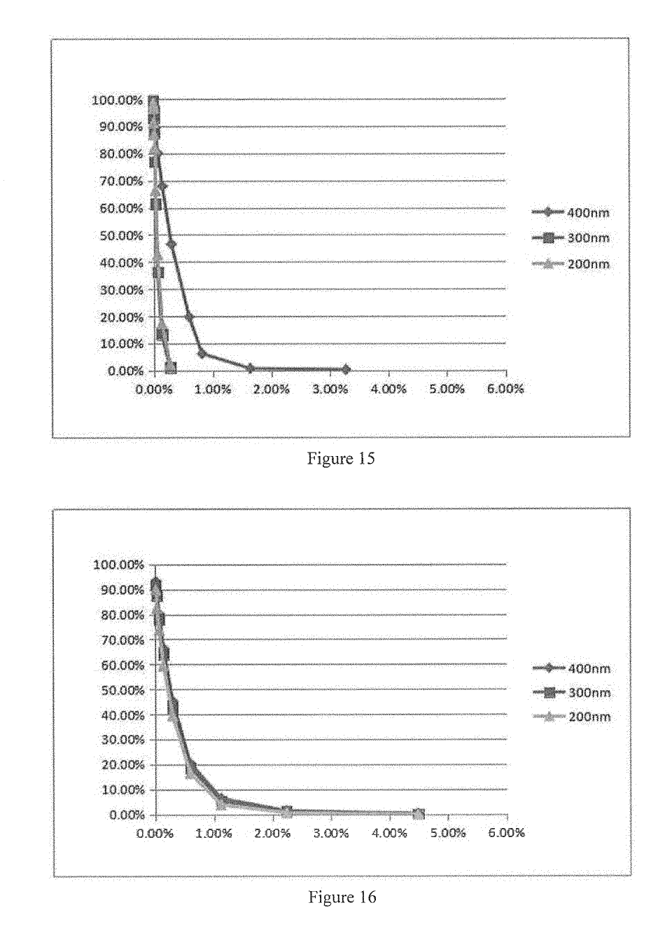

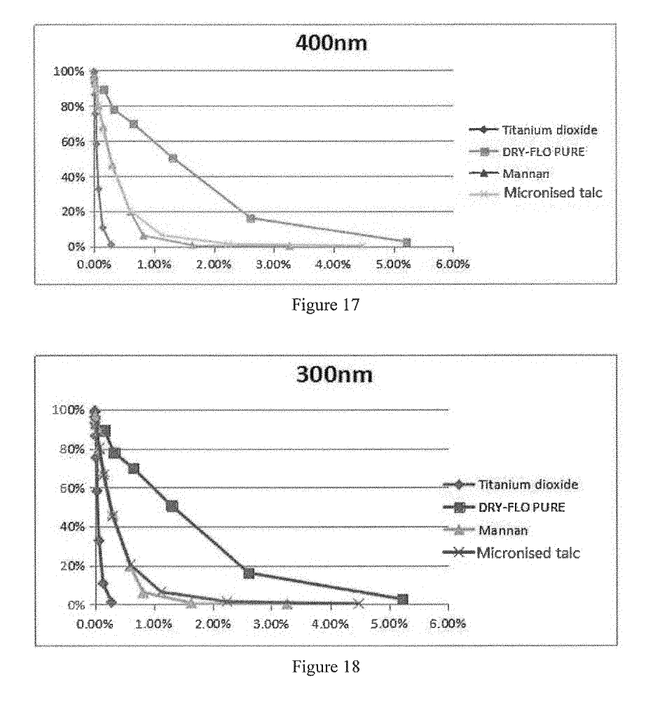

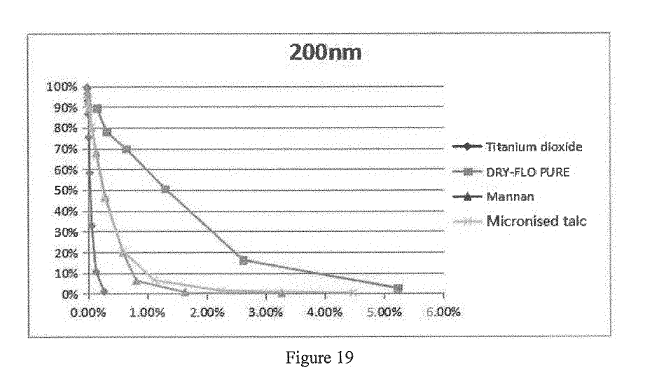

FIGS. 13 through 19 provide transmittance values for the above materials.

According to above data, titanium dioxide had the best UV shielding ability. The modified starch (DRY-FLO PURE) had the worst UV shielding ability. Around 50% transmittance, Mannan requires 5-6 times the amount of Titanium dioxide to achieve the similar effect.

Ivory nut powder for the above measurements and scanning electron micrographs (SEMs) was prepared by physical processing only, so the surface is similar to ivory nut. Mannan was extracted from ivory nut powder, and compared to SEMs of the ivory nut powder to note differences in morphology.

Preparation Method of Ivory Nut Powder (200 Mesh):

Ivory nuts (whole nut without dark peel) were crushed to particles by a jaw crusher at room temperature. The particles were pulverized to a powder (95% through 200 mesh sieve) by use of a universal pulverizer. The particle size was controlled via adjustments to the universal pulverizer parameters.

Preparation Method of Mannan (D95≤10 μm, D50≤55 μm):

Sodium hydroxide and deionized water were mixed to prepare a clear alkali solution as described below.

To the sodium hydroxide solution maintained at 20° C. was slowly added ivory nut as described above. The mixture was maintained at 20° C. and stirred for 2 hour. The solids were then filtered, treated a second time as described with sodium hydroxide for 2 hours and filtered. The filtrates from the two filtrations were combined to provide a homogenous solution. The pH of the filtrate solution was adjusted to a pH of 4.0 with stirring and allowed to remain for 12 hours with the formation of a precipitate.

The precipitated solid was filtered, dried, collected and pulverized into powder with a Pneumatic pulverizer. The particle size of the powder was controlled with Pneumatic pulverizer to D95≤10 μm, D50≤5 μm. That obtain a kind of white powder (as described below) with a smooth feel equivalent to talcum powder.

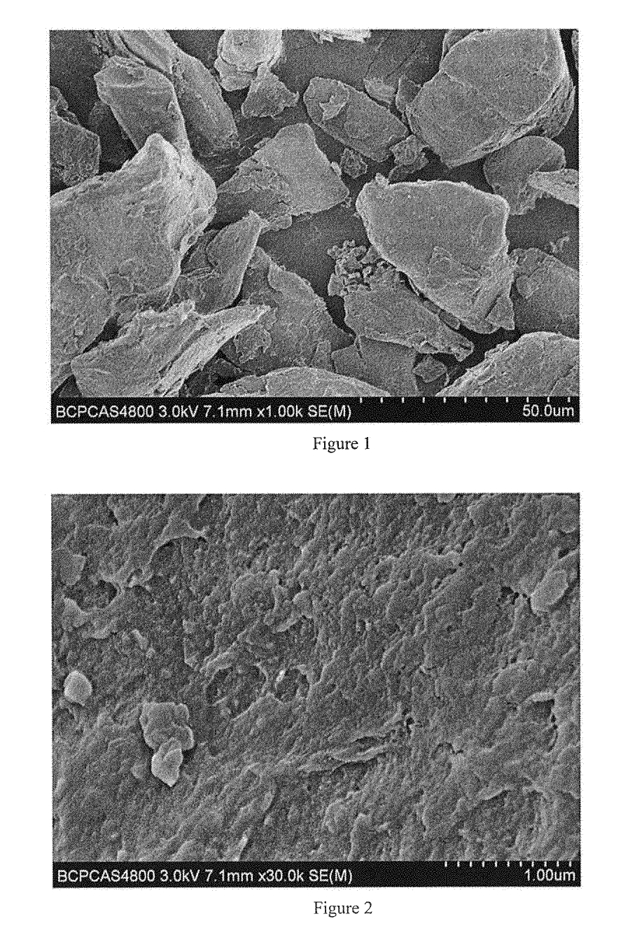

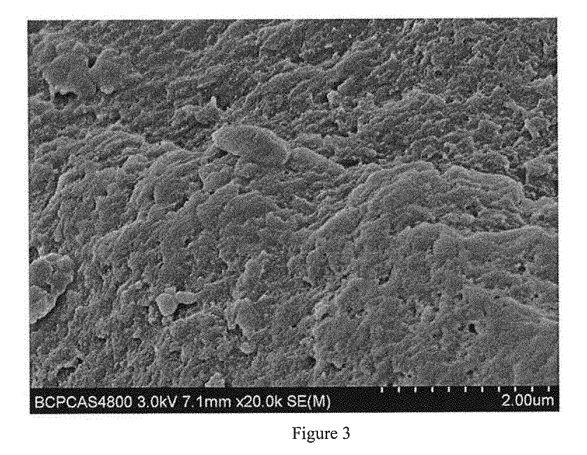

FIGS. 1 through 3 are SEM photographs of the morphology of the ivory nut particulate surface as described above. The micrographs of the ivory nut powder (200 mesh) showed that it is irregular blocks less than 50 microns. The micrographs depict a multitude of micropores on the surface of the blocks.

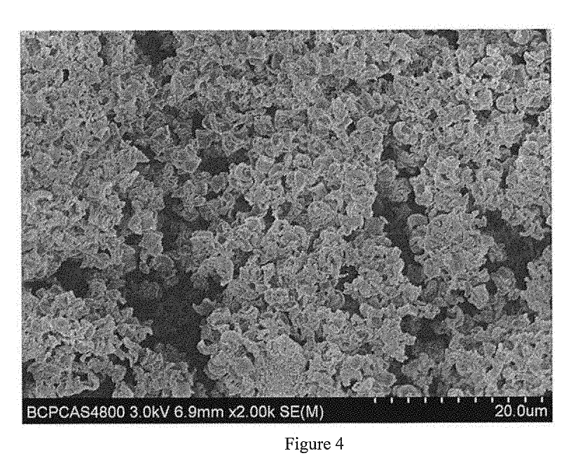

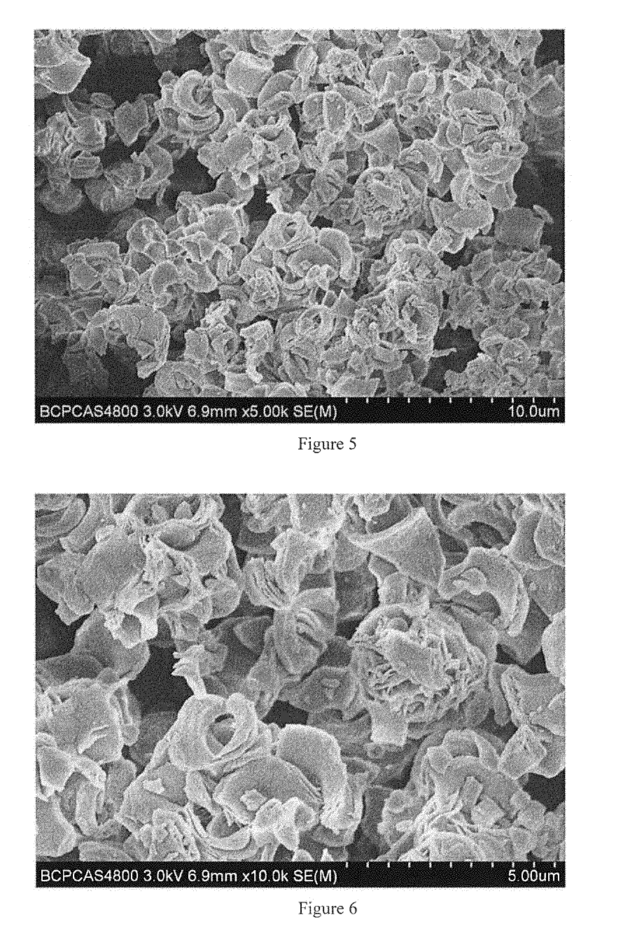

FIGS. 4 through 6 are SEM photographs of the morphology of the mannan particulate surface as described above. The micrographs of the mannan (D95≤10 μm, D50≤5 μm) showed it is flake (layer type) and most of it less than 5 microns. FIG. 6 provides that the flake is composed of a many nanoscale layered solid. Not to be bound by theory, this special structure may cause the mannan particulate feel smooth to the touch. (D95 represents 95 percent (w/w), D50 represent 50 percent (w/w), etc.

### EXAMPLES: THE PREPARATION OF ULTRAFINE IVORY NUT POWDER PRODUCTS

### Example 1

Raw Materials:

| Raw material name | Specification | Quantity | |
|---|---|---|---|
| Ivory nut | Whole nut without dark peel | 500 | g |
| Deionzed water | None | 1000 | ml |

Preparation Method:

Ivory nuts (whole nut without dark peel) were crushed to particles by a jaw crusher at room temperature. The particles were pulverized to powder (95% through 100 mesh sieve) with a universal pulverizer. The particle size was controlled to be 95% through 10-120 mesh seive via adjustments to the universal pulverizer parameters.

The powder was then milled to ultrafine powder (90%≤5 μm, 50%≤2 μm) by use of a wet ball mill. The particle size of ultrafine powder was controlled via the controlling of milling time.

Reflectivity Test

Detection instrument: TU-1901+60 Integrating sphere

Experimental Conditions:

Scanning range: 850-230 nm

Scanning speed: Medium speed

Spectral bandwidth: 5 nm

Measurement method: R %

The Test Results:

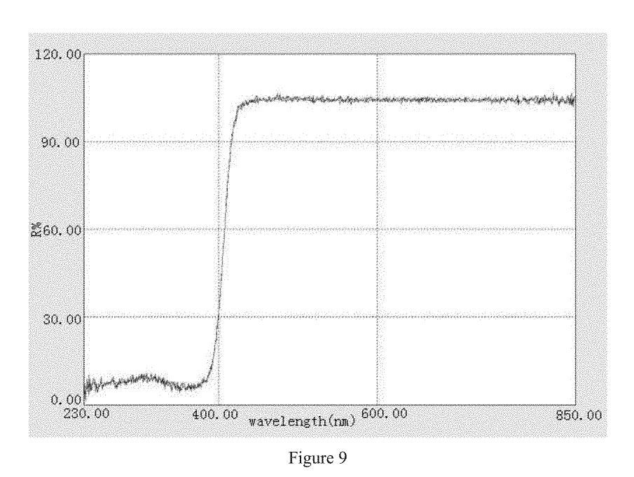

The reflectivity of the above ivory nut powder was compared with titanium dioxide powder (particle size 0.26-0.42 microns). The results showed that the reflectivity of the ivory nut powder was much higher than the titanium dioxide powder below 400 nm. The reflectivity of ivory nut powder was lower than titanium dioxide powder above 400 nm. (FIG. 9)

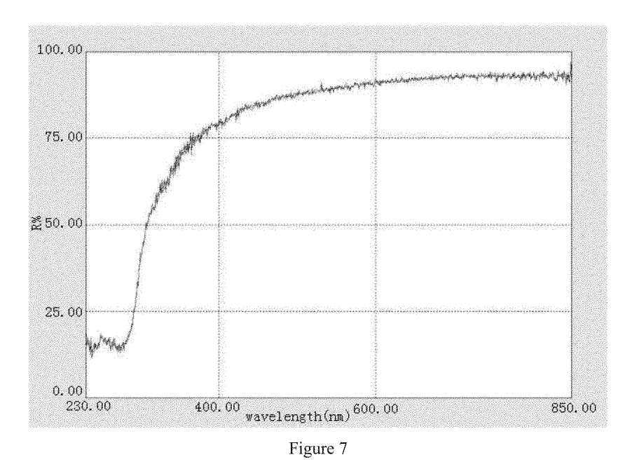

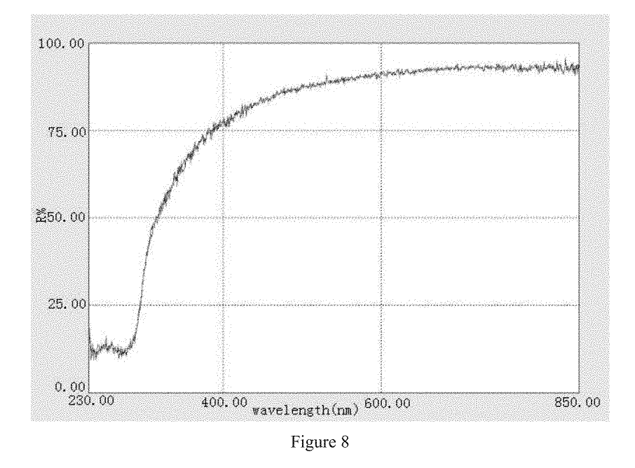

The reflectivity of ivory nut powder (particle sizes of 95% through 100 mesh sieve) (FIG. 7) is better than ivory nut powder (particles sizes were 95% through 80 mesh sieve not through 100 mesh sieve: less than or equal to 200 microns but greater than or equal to 150 microns) (FIG. 8). Thus, finer particle sizes resulted in higher reflectivity.

Advantageously, ivory nut powder is excellent for UVB protection and can be used for sunscreen protection.

### Example 2

Preparation of Superfine Mannan

| Raw material name | Specification | Quantity | |
|---|---|---|---|
| Sodium hydroxide | AR | 400 | g |
| Ivory nut powder | Through 80 mesh sieve | 500 | g |
| | Not through 100 mesh sieve | | |
| Deionzed water | None | 4000 | ml |

Preparation Method of Mannan (300 Mesh) (as Noted Above):

400 g sodium hydroxide and 4000 ml deionized water were mixed to prepare a clear alkali solution.

To the sodium hydroxide solution maintained at 20° C. was slowly added 500 g of ivory nut. The mixture was maintained at 20° C. and stirred for 2 hour. The solids were then filtered, treated a second time as described with sodium hydroxide for 2 hours and filtered. The filtrates from the two filtrations were combined to provide a homogenous solution. The pH of the filtrate solution was adjusted to a pH of 4.0 with stirring and allowed to remain for 12 hours.

The precipitated solid was filtered, dried (vacuum with heat or spray dried) collected and pulverized into powder with a Pneumatic pulverizer. The powder was sieved with a 300 mesh sieve (particle size is 50%≤55.0 μm and 95%≤10 μm) to obtain a white powder with a smooth feel equivalent to talcum powder.

Conclusion: The superfine mannan powder can be used for cosmetic ingredients to replace or mix with talc, such as in a cooling powder etc.

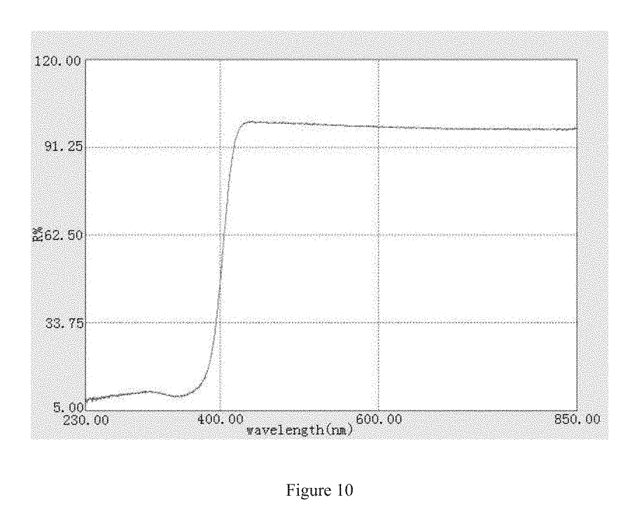

The powdered mannan from above was compared to titanium dioxide (particle size 0.26-0.42 μm) as noted in FIGS. 10 through 12.

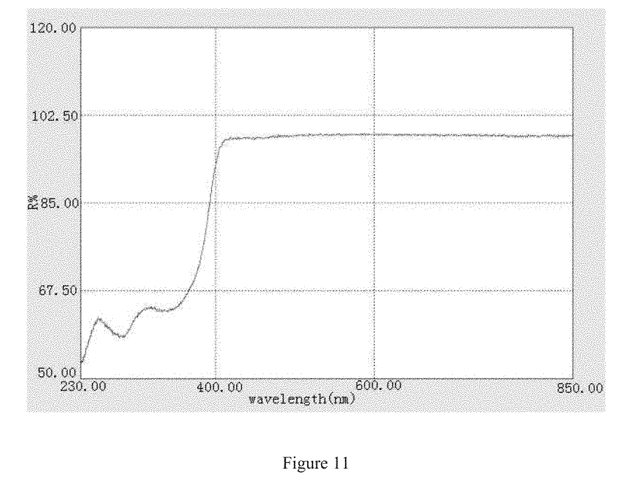

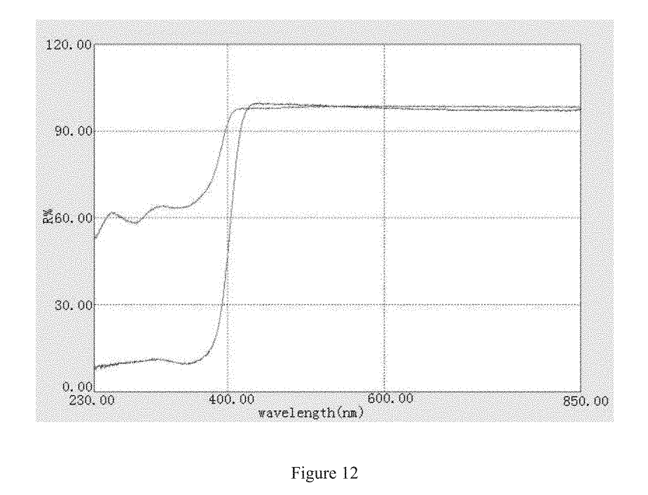

Results: The reflectivity of mannan was compared with titanium dioxide powder. The results showed that the reflectivity of mannan is much higher than titanium dioxide powder below 400 nm. The results also showed that mannan has a much better UV shielding ability than titanium dioxide (FIG. 12). Because titanium dioxide has a strong ability to absorb UV energy (200-400 nm) such absorption can create a lot of free radicals via photochemical reactions (FIG. 10). This causes damage to the skin. Mannan has a high reflectivity at 200-400 nm which means mannan has strong ability of physical UV shielding (FIG. 11). Therefore, when compared to titanium dioxide, mannan is much safer than titanium dioxide in the ultraviolet band region. The reflectivity of mannan is the same as titanium dioxide above 400 nm. Both of them have a great ability for visible light shielding (FIG. 12). The particle size distribution of Micronized Talc is 98%≤9 μm, 50%≤2.2 μm.

Stability Tests of Examples of Mannan and Other Similar Products

| | Sample | Producer/ | Art.-Nr./Lot- | |
|---|---|---|---|---|
| Product Name | Code | Brand | Nr./Batch Nr. | Purchased |
| EPCannan | 18295 | EPC | 20170604 | EPC |
| (Mannan) | | | | |
| Micronized Talc | 18296 | Mondo | 20170604 | |
| | | Minerals | | |
| Corn Starch | 18297 | Maizena | L415304804 | Billa |
| Novelose 330 | 18323 | National | MAI6659 | National |
| (High amylose | | Starch | | Starch |
| maize starch) | | | | |

Heat Stability

The samples were treated for 30 min or respectively 15 h and the changes afterwards measured with a Color Quest Spectrophotometer against an untreated sample.

| | | L* | a* | b* | dL* | da* | db* | dE* |
|---|---|---|---|---|---|---|---|---|
| 18295 | Standard | 87.22 | −0.76 | 1.15 | | | | |
| 18295 | 90° C./30 min | 87.12 | −0.75 | 1.18 | −0.10 | 0.01 | 0.03 | 0.10 |
| 18295 | 90° C./15 h | 87.12 | −0.76 | 1.31 | −0.09 | −0.01 | 0.16 | 0.18 |
| 18296 | Standard | 88.05 | −0.66 | 0.38 | | | | |
| 18296 | 90° C./30 min | 88.27 | −0.62 | 0.66 | 0.22 | 0.04 | 0.28 | 0.36 |
| 18296 | 90° C./15 h | 88.04 | −0.62 | 0.61 | −0.01 | 0.04 | 0.23 | 0.23 |
| 18297 | Standard | 89.21 | −1.18 | 4.66 | | | | |
| 18297 | 90° C./30 min | 89.04 | −1.23 | 4.75 | −0.18 | −0.05 | 0.08 | 0.20 |
| 18297 | 90° C./15 h | 88.84 | −1.20 | 4.73 | −0.37 | −0.02 | 0.07 | 0.38 |
| 18323 | Standard | 87.33 | −0.47 | 5.49 | | | | |
| 18323 | 90° C./30 min | 86.94 | −0.48 | 5.55 | −0.39 | −0.01 | 0.06 | 0.39 |
| 18323 | 90° C./15 h | 86.91 | −0.53 | 6.39 | −0.42 | −0.06 | 0.91 | 1.00 |

UV Stability

The samples were treated with UV Light for 1 h or respectively 15 h and the changes afterwards measured with a Color Quest Spectrophotometer against an untreated sample.

| | | L* | a* | b* | dL* | da* | db* | dE* |
|---|---|---|---|---|---|---|---|---|
| 18295 | Standard | 87.22 | −0.76 | 1.15 | | | | |
| 18295 | UV/1 h | 87.48 | −0.71 | 1.21 | 0.26 | 0.04 | 0.06 | 0.27 |
| 18295 | UV/15 | 87.37 | −0.73 | 1.23 | 0.16 | 0.03 | 0.08 | 0.18 |
| 18296 | Standard | 88.05 | −0.66 | 0.38 | | | | |
| 18296 | UV/1 h | 88.21 | −0.65 | 0.61 | 0.16 | 0.01 | 0.23 | 0.28 |
| 18296 | UV/15 | 88.18 | −0.61 | 0.67 | 0.13 | 0.06 | 0.29 | 0.32 |
| 18297 | Standard | 89.21 | −1.18 | 4.66 | | | | |
| 18297 | UV/1 h | 89.20 | −1.22 | 4.73 | −0.01 | −0.04 | 0.07 | 0.08 |
| 18297 | UV/15 | 89.34 | −1.21 | 4.79 | 0.13 | −0.03 | 0.13 | 0.19 |
| 18323 | Standard | 87.33 | −0.47 | 5.49 | | | | |
| 18323 | UV/1 h | 87.27 | −0.49 | 5.49 | −0.07 | −0.02 | 0.01 | 0.07 |
| 18323 | UV/15 | 87.27 | −0.45 | 5.43 | −0.06 | 0.01 | −0.05 | 0.08 |

All 4 types of products (Mannan, Talc, Starch and processed starch) have a very good UV and heat stability similar to each other.

Stability Tests of Examples of Dyed Tagua (Ivory Nut) Powder

pH Stability

Samples of dyed ivory nut powder prepared as described in Examples 6, 7 and 8 were dispersed in 10 ml of ammonium acetate buffer over a pH range of from 2 to 10 at room temperature for 24 hours. No change in color loss was noted. The samples were stable over the pH range of 2 to 10.

Heat Stability

Samples of dyed ivory nut powder prepared as described in Examples 5, 6 and 8 were subjected to 90° C. in a cabinet dryer for 30 minutes and 15 hours. The “standard” was a sample of each example that was not subjected to the increased temperature and remained at ambient conditions. Color differences were measured with a ColorQuest Photometer.

| | L* | a* | b* | dL* | da* | db* | dE* |
|---|---|---|---|---|---|---|---|
| Green | | | | | | | |
| Standard | 43.66 | −6.27 | 4.13 | 43.66 | −6.27 | 4.13 | |
| 90° C./30 min | 43.13 | −5.82 | 3.88 | −0.53 | 0.46 | −0.25 | 0.74 |
| 90° C./15 h | 43 | −5.5 | 3.85 | −0.67 | 0.78 | −0.27 | 1.06 |
| Yellow | | | | | | | |
| Standard | 63.67 | 25.92 | 34.66 | 3.67 | 25.92 | 34.66 | |
| 90° C./30 min | 79.7 | 0.13 | 25.72 | 16.03 | −25.79 | −8.94 | 31.65 |
| 90° C./15 h | 81.01 | −0.77 | 17.34 | 17.34 | −26.69 | −17.32 | 36.24 |
| Red | | | | | | | |
| Standard | 49.12 | 29.93 | 19.56 | 49.12 | 29.93 | 19.56 | |
| 90° C./30 min | 48.94 | 30.19 | 20.62 | −0.18 | 0.26 | 1.06 | 1.1 |
| 90° C./15 h | 57.57 | 29.53 | 32.74 | 8.6 | −0.39 | 13.17 | 15.66 |

Photometer: Hunter Lab ColorQuest XE

Method: Reflectance Specular Included

Color Scale: Hunter Lab Scale (for more information see attached file ColorQuest XE_Manual page 10-1)

dE is the sum of the color difference between the untreated and the treated sample. If dE is smaller the changes are slighter. If it is larger the changes are higher. Color differences with a dE above 2.0 can be seen with naked eyes.

Conclusions:

Tagua Powder Red and Green: very good short term heat stability, can be added to the final product at higher temperatures.

Tagua Powder Yellow: should be added at room temperature to avoid loss of color from heating processes.

Samples showed good short term heat stability.

UV Stability

Samples of dyed ivory nut powder prepared as described in Examples 5, 6 and 8 were subjected to UV light at 253.7 nm for 60 minutes and 15 hours. The “standard” was a sample of each example that was not subjected to the UV treatment and remained at ambient conditions. Color differences were measured with a ColorQuest Photometer.

| | L* | a* | b* | dL* | da* | db* | dE* |
|---|---|---|---|---|---|---|---|
| Green | | | | | | | |
| UV/1 h | 43.38 | −8.24 | 5.88 | 43.38 | −8.24 | 5.88 | |
| UV/15 h | 43.14 | −7.38 | 5.83 | −0.24 | 0.87 | −0.06 | 0.9 |
| Standard | 45.05 | −4.49 | 6.61 | 1.68 | 3.76 | 0.73 | 4.18 |
| Yellow | | | | | | | |
| UV/1 h | 67.69 | 30.27 | 42.66 | 67.69 | 30.27 | 42.66 | |
| UV/15 h | 69.17 | 27.57 | 36.91 | 1.48 | −2.7 | −6.75 | 7.42 |
| Standard | 75.3 | 16.42 | 18.26 | 7.61 | −13.85 | −24.4 | 29.07 |
| Red | | | | | | | |
| UV/1 h | 49.96 | 37.28 | 24.78 | 49.96 | 37.28 | 24.78 | |
| UV/15 h | 55.58 | 30.68 | 22.31 | 5.62 | −6.6 | −2.47 | 9.01 |
| Standard | 76.66 | 7.53 | 9.34 | 26.7 | −29.75 | −15.44 | 42.85 |

Very good short term UV stability for all samples. As a precaution, they can be stored in a dark place to ensure long term stability.

Samples showed good short UV stability.

Stability tests of Examples of Dyed Tagua Powder in Formulations

0.3 g samples of dyed ivory nut powder prepared as described in Examples 5, 6 and 8 were mixed in 100 g of Nivea Body Lotion “Happy Time” (white) and 100 g Basis pH shower gel subjected to 90° C. conditions or UV light at 253.7 nm for 15 hours. The “standard” was a sample of each example that was not subjected to the elevated temperature and UV treatment and remained at ambient conditions. Color differences were measured with a ColorQuest Photometer.

Results for Body Lotion

| | L* | a* | b* | dL* | da* | db* | dE* |
|---|---|---|---|---|---|---|---|
| Green | | | | | | | |
| Standard | 75.19 | −3.62 | 1.74 | 75.19 | −3.62 | 1.74 | |
| 90° C./ | 71.47 | −3.93 | 1.56 | −3.72 | −0.31 | −0.18 | 3.74 |
| 15 h | | | | | | | |
| UV/15 h | 74.24 | −3.68 | 1.73 | −0.96 | −0.06 | −0.01 | 0.96 |
| Yellow | | | | | | | |
| Standard | 79.21 | 2.57 | 4.35 | 79.21 | 2.57 | 4.35 | |
| 90° C./ | 76.78 | 2.33 | 3.68 | −2.43 | −0.23 | −0.67 | 2.53 |
| 15 h | | | | | | | |
| UV/15 h | 77.45 | 2.16 | 3.63 | −1.76 | −0.4 | −0.72 | 1.94 |
| Red | | | | | | | |
| Standard | 75.28 | 7.03 | 5.3 | 75.2.8 | 7.03 | 5.3 | |
| 90° C./ | 74.5 | 5.19 | 3.83 | −0.77 | −1.83 | −1.47 | 2.47 |
| 15 h | | | | | | | |
| UV/15 h | 75.78 | 3.79 | 2.77 | 0.51 | −3.24 | −2.53 | 4.14 |

Results for Shower Gel

| | L* | a* | b* | dL* | da* | db* | dE* |
|---|---|---|---|---|---|---|---|
| Green | | | | | | | |
| Standard | 80.82 | −6.01 | 9.38 | 80.82 | −6.01 | 9.38 | |
| 90° C./ | 86.4 | −8.23 | 10.94 | 5.58 | −2.22 | 1.56 | 6.21 |
| 15 h | | | | | | | |
| UV/15 h | 77.99 | −6.68 | 10.51 | −2.83 | −0.68 | 1.13 | 3.12 |
| Yellow | | | | | | | |
| Standard | 83.94 | 0.91 | 5.42 | 83.94 | 0.91 | 5.42 | |
| 90° C./ | 88.45 | −2.43 | 21.54 | 4.51 | −3.35 | 16.12 | 17.07 |
| 15 h | | | | | | | |
| UV/15 h | 82.29 | 1.32 | 5.87 | −1.65 | 0.41 | 0.45 | 1.76 |
| Red | | | | | | | |
| Standard | 74.99 | 14.36 | 69.28 | 74.99 | 14.36 | 69.28 | |
| 90° C./ | 78.28 | 21.93 | 83.24 | 3.29 | 7.56 | 13.96 | 16.22 |
| 15 h | | | | | | | |
| UV/15 h | 73.99 | 14.77 | 68.16 | −1 | 0.41 | −1.12 | 1.55 |

Samples had very good long term UV and heat stability.

### Additional Examples of Baby Powder Formulations with Mannan

### Example 1

Raw Materials:

| Raw material name | Specification | Quantity | |
|---|---|---|---|
| Sodium hydroxide | AR | 400 | g |
| Ivory nut powder | Through 80 mesh sieve | 500 | g |
| | Not through 100 mesh sieve | | |
| Deionzed water | None | 4000 | ml |

Preparation Method:

Sodium hydroxide and deionized water were mixed to provide a clear alkali solution. To the solution with stirring, while maintaining the temperature at less than 20° C., the ivory nut was slowly added over a 2 hour period. Solids were filtered and filtrate was retained. The solids were subjected to a second treatment with an alkaline solution as described above and filtered. The retentates were combined and mixed to provide a homogenous solution. The pH of the solution was adjusted to a pH value of 4.0 with stirring. The solution/resultant precipitate was allowed to stand for 12 hours at ambient temperature. The solids were collected via filtration and washed several times with water until the pH of the rinse solution was between about 6.5 to about 7.5. The solids were dried, pulverized with a universal pulverizer and sieved with a 300 mesh sieve to obtain a white powder with a smooth feel similar to that of talcum powder.

### Example 2

Raw Materials:

| Raw material name | Specification | Quantity | |
|---|---|---|---|
| Sodium hydroxide | AR | 400 | g |
| Ivory nut powder | Through 80 mesh sieve | 500 | g |
| | Not through 100 mesh sieve | | |
| Deionzed water | None | 4000 | ml |

Preparation Method:

Sodium hydroxide and deionized water were mixed to provide a clear alkali solution. To the solution with stirring, while maintaining the temperature at less than 20° C., the ivory nut was slowly added over a 2 hour period. Solids were filtered and filtrate was retained. The solids were subjected to a second treatment with an alkaline solution as described above and filtered. The retentates were combined and mixed to provide a homogenous solution. The pH of the solution was adjusted to a pH value of 4.0 with stirring. The solution/resultant precipitate was allowed to stand for 12 hours at ambient temperature. The solids were collected via filtration and washed several times with water until the pH of the rinse solution was between about 6.5 to about 7.5. The solids were spray dried to provide a white powder with a particle size of 95% of particles being less than or equal to 10 microns.

### EXAMPLES OF MANNAN FROM IVORY NUT

### Example 1

Preparation Method:

Mannan (as prepared above) was sieved with a 200 mesh sieve. A white uniform powder was obtained.

Natural baby powder formula:

| Ingredient name | Quantity/100 g |
|---|---|
| Mannan from ivory nut | 100.0 g |

### Example 2

Preparation Method:

Mannan, Zinc stearate, Magnesium stearate and orange essence were added into a mixer and mixed until a homogenous mixture was obtained. The mixture was sieved with a 200 mesh sieve to afford a white powder.

Natural Baby Powder Formula:

| Ingredient name | Quantity/100 g | |
|---|---|---|
| Mannan from ivory nut | 97.50 | g |
| Zinc stearate | 0.50 | g |
| Magnesium stearate | 1.00 | g |
| Orange Essence | 1.00 | g |

### Example 3

Preparation Method:

Mannan and Corn starch were added into a mixer and mixed until a homogenous mixture was obtained. Zinc stearate, Magnesium stearate and orange essence were added to the mixture and mixed until a homogenous mixture was obtained. The mixture was sieved with a 200 mesh sieve to afford a white powder.

Natural Baby Powder Formula:

| Ingredient name | Quantity/100 g | |
|---|---|---|
| Mannan from ivory nut | 50.00 | g |
| Corn starch | 47.50 | g |
| Zinc stearate | 0.50 | g |
| Magnesium stearate | 1.00 | g |
| Orange Essence | 1.00 | g |

### Example 4

Preparation Method:

Mannan, Corn starch and Cassava starch were added into a mixer and mixed until a homogenous mixture was obtained. Zinc stearate, Magnesium stearate and orange essence were added to the mixture and mixed until a homogenous mixture was obtained. The mixture was sieved with a 200 mesh sieve to afford a white powder.

Natural Baby Powder Formula:

| Ingredient name | Quantity/100 g | |
|---|---|---|
| Mannan from ivory nut | 50.00 | g |
| Corn starch | 27.50 | g |
| Cassava starch | 20.00 | g |
| Zinc stearate | 0.50 | g |
| Magnesium stearate | 1.00 | g |
| Orange Essence | 1.00 | g |

### Example 5

Preparation Method:

Mannan, Corn starch and Cassava starch were added into a mixer and mixed until a homogenous mixture was obtained. Purslane, Zinc stearate, Magnesium stearate and orange essence were added to the mixture and mixed until a homogenous mixture was obtained. The mixture was sieved with a 200 mesh sieve to afford a white powder.

Natural Baby Powder Formula:

| Ingredient name | Quantity/100 g | |
|---|---|---|
| Mannan from ivory nut | 50.00 | g |
| Corn starch | 27.50 | g |
| Cassava starch | 18.00 | g |
| Purslane extract | 2.00 | g |
| Zinc stearate | 0.50 | g |
| Magnesium stearate | 1.00 | g |
| Orange Essence | 1.00 | g |

### Example 6

Preparation Method:

Mannan, Corn starch and Cassava starch were added into a mixer and mixed until a homogenous mixture was obtained. Bitter Melon, Purslane, Zinc stearate, Magnesium stearate and orange essence were added to the mixture and mixed until a homogenous mixture was obtained. The mixture was sieved with a 200 mesh sieve to afford a white powder.

Natural Baby Powder Formula:

| Ingredient name | Quantity/100 g | |
|---|---|---|
| Mannan from ivory nut | 50.00 | g |
| Corn starch | 25.50 | g |
| Cassava starch | 18.00 | g |
| Purslane extract | 2.00 | g |
| Bitter melon extract | 2.00 | g |
| Zinc stearate | 0.50 | g |
| Magnesium stearate | 1.00 | g |
| Orange Essence | 1.00 | g |

### Example 7

Preparation Method:

Mannan, Corn starch and Cassava starch were added into a mixer and mixed until a homogenous mixture was obtained. Mint, Bitter Melon, Purslane, Zinc stearate, Magnesium stearate and orange essence were added to the mixture and mixed until a homogenous mixture was obtained. The mixture was sieved with a 200 mesh sieve to afford a white powder.

Natural Baby Powder Formula:

| Ingredient name | Quantity/100 g | |
|---|---|---|
| Mannan from ivory nut | 50.00 | g |
| Corn starch | 25.00 | g |
| Cassava starch | 17.00 | g |
| Purslane extract | 2.00 | g |
| Bitter melon extract | 2.00 | g |
| Mint | 0.50 | g |
| Zinc stearate | 0.50 | g |
| Magnesium stearate | 1.00 | g |
| Orange Essence | 1.00 | g |

### Example 8

Preparation Method:

Mannan, Corn starch and Cassava starch were added into a mixer and mixed until a homogenous mixture was obtained. Mint, Bitter Melon, Purslane, Tea extract, Ferulic acid, Zinc stearate, Magnesium stearate and orange essence were added to the mixture and mixed until a homogenous mixture was obtained. The mixture was sieved with a 200 mesh sieve to afford a white powder.

Natural Baby Powder Formula:

| Ingredient name | Quantity/100 g | |
|---|---|---|
| Mannan from ivory nut | 50.00 | g |
| Corn starch | 24.00 | g |
| Cassava starch | 17.00 | g |
| Purslane extract | 2.00 | g |
| Bitter melon extract | 2.00 | g |
| Tea extract | 0.50 | g |
| Ferulic acid | 0.50 | g |
| Mint | 0.50 | g |
| Zinc stearate | 0.50 | g |
| Magnesium stearate | 1.00 | g |
| Orange Essence | 1.00 | g |

### Example 9

Preparation Method:

The A phase (noted in the table below) was added to a reaction kettle with agitation for 5 min. The B phase was added to the A phase mixture and heated to 80° C. and maintained for 10 minutes until the mixture was completely dissolved. Each component of the C phase was added to the mixture individually with agitation and heated until the mixture was uniformly dispersed at 75° C. The D phase was added with agitation. The mixture was placed under vacuum under −0.06 Mpa and kept at 75° C. for 10 minute to discharge any air bubbles. The mixture was cooled down to 35° C. and poured into molds and formed.

Lipstick Formula:

| | |
|---|---|
| Ingredient name | Quantity/100 g |

A phase

| | | |
|---|---|---|
| White oil | 34.00 | g |
| Aerosil | 1.00 | g |

B phase

| | | |
|---|---|---|
| Beewax | 8.40 | g |
| Candelilla wax | 1.70 | g |
| Microcrystalline wax | 3.70 | g |
| Polyisobutylene | 35.15 | g |

C phase

| | | |
|---|---|---|
| Ivory nut powder yellow | 2.96 | g |
| Mannan from ivory nut | 7.30 | g |
| Ivory nut powder red | 2.16 | g |
| Iron oxide black | 0.13 | g |

D phase

| | | |
|---|---|---|
| Pearl powder | 3.50 | g |

Ivory nut powder yellow was dyed with gardenia yellow. Ivory nut powder red was dyed with gardenia red

### Example 10

Preparation Method:

Superfine gardenia red dyed ivory nut powder was premixed in castor oil (part D) at room temperature to ensure uniformity.

A hot plate was heated to 75 centigrade.

All part C ingredients were melted in a separate beaker on the hot plate and the mixture was blended by hand.

Heating was discontinued and parts A and B were added to C and mixed.

Part D was then added to the mixture until the color was uniform. Part E was then added to the mixture and the mixture was mixed slowly to remove air bubbles.

Lipstick molds were filled with the mixture, chilled for 10 minutes at −6 degrees centigrade and transferred into individual cases.

Lipstick Formula:

| | |
|---|---|
| Ingredient name | Quantity/100 g |

A phase

| | | |
|---|---|---|
| Castor Oil | 7.50 | g |
| Grape Seed Oil | 8.50 | g |

B phase

| | | |
|---|---|---|
| Sucrose Acetate Isobutyrate | 16.00 | g |
| Triisostearyl citrate | 8.80 | g |

C phase

| | | |
|---|---|---|
| Cetyl Palmitate | 4.40 | g |
| Beewax | 4.40 | g |
| Candelilla Wax | 5.30 | g |
| Pracera C44 | 2.60 | g |

D phase

| | | |
|---|---|---|
| Superfine Gardenia Red Dyed Ivory Nut powder | 7.00 | g |
| Castor Oil | 35.00 | g |

E phase

| | |
|---|---|
| Vitamin E | 0.50% |

The particle size of Superfine Gardenia Red Dyed Ivory Nut powder was 90%≤10 μm.

### Example 11

Preparation Method:

Mannan and borneol were added into mixer and mixed until a homogenous mixture was obtained. Green tea extract, Purslane extract, Ferulic acid, orange essence were added to the mixture and mixed until the contents became a homogeneous mixture. The mixture was sieved with a 200 mesh sieve to afford a yellow powder.

Deodorant Powder Formula:

| Ingredient name | Quantity/100 g | |
|---|---|---|
| Mannan from ivory nut | 50.00 | g |
| Green tea extract | 10.00 | g |
| Purslane extract | 30.00 | g |
| Ferulic acid | 7.00 | g |
| Borneol | 2.00 | g |
| Orange essence | 1.00 | g |

### Example 12

Preparation Method:

The B phase was added to a reaction kettle with agitation until the mixture was completely dissolved. The A phase components were individually added to the B phase mixture with agitation and heated at 80° C. until a uniform mixture was obtained. The C phase was added to a second reaction kettle with agitation and heated until the mixture was completely dissolved at room temperature. The C phase was added to emulsifying kettle with stirring for 10 minutes and heated to 90° C. Then the mixture was added to the emulsifying kettle and homogenized for 6 minutes at 2500 rpm and then stirred for 20 minutes at 80° C. The mixture was cooled down to 60° C. and the D phase was added to the emulsion with agitation for 2 minutes followed by homogenization for 2 minutes at 2500 rpm. The E phase was added to the mixture and stirred for 10 minutes. The final mixture was filtered and a final lotion product was obtained.

Sunscreen Lotion Formula:

| | |
|---|---|
| Ingredient name | Quantity/100 g |

A phase

| | | |
|---|---|---|
| C12-20 Alkyl Polyglycoside | 2.50 | g |
| Silicone oil | 1.00 | g |
| Stearic acid | 0.50 | g |
| Stearic acid glycol ester | 0.50 | g |
| C16-18 alcohol | 1.50 | g |
| Nipasol | 0.20 | g |
| Mannan from ivory nut | 10.00 | g |
| Green Tea Polyphenols Palmitate | 5.00 | g |
| Oryzanol | 1.00 | g |
| Alkyl polyvinyl ketone | 2.00 | g |
| C12-15 alkyl benzoate | 2.00 | g |

B phase

| | | |
|---|---|---|
| Octyl methoxycinnamate | 7.00 | g |
| Diphenyl ketone | 2.50 | g |
| Octyl salicylate | 2.50 | g |

C phase

| | | |
|---|---|---|
| Clycerol | 6.50 | g |
| Veegum | 0.25 | g |
| Xanthan gum | 0.20 | g |
| Laurel potassium | 0.35 | g |
| Deionized water | 54.17 | g |

D phase

| | | |
|---|---|---|
| Menthyl Lactate | 0.10 | g |
| Safflower Oleosome | 3.30 | g |

E phase

| | | |
|---|---|---|
| Green apple essence | 0.15 | g |
| Isothiazolinones | 0.08 | g |

### Example 13

Preparation Method:

Mannan, Ivory nut powder red, Ivory nut powder yellow and Iron oxide black were added into a mixer, and mixed until a homogenous mixture was obtained. To the mixture was then added Isotridecyl alcohol, Isononanoic acid ester, Phenyl Trimethicone, Zinc stearate and lavender Essence with mixing until a homogenous mixture was obtained. The mixture was sieved with a 200 mesh sieve to afford a light orange red powder.

Loose Powder Formula:

| Ingredient name | Quantity/100 g | |
|---|---|---|
| Mannan from ivory nut | 62.00 | g |
| Ivory nut powder red | 12.00 | g |
| Ivory nut powder yellow | 10.00 | g |
| Iron oxide black | 3.00 | g |
| Isotridecyl alcohol Isononanoic acid ester | 5.00 | g |
| Phenyl Trimethicone | 5.50 | g |
| Zinc stearate | 0.50 | g |
| Magnesium stearate | 1.00 | g |
| Lavender Essence | 1.00 | g |

### Example 14

Preparation Method:

The A phase and C phase were added to a reaction kettle with agitation until the mixture was uniformly dispersed. The B phase was added to the mixture with agitation and heated to 85° C. The D phase was added to the mixture with agitation, stirred for 15 minutes and heated at 80° C. While the mixture was stirred, the temperature was reduced to 50° C. The warmed mixture was filled into eye shadow containers.

Eye Shadow Formula:

| | |
|---|---|
| Ingredient name | Quantity/100 g |

A phase

| | | |
|---|---|---|
| Phenyl Silicone Oil | 9.00 | g |
| Silicone polyether | 4.00 | g |

B phase

| | | |
|---|---|---|
| Potassium cetyl phosphate/ | 10.55 | g |
| hydrogenated palm glycerides | | |
| Bee gum | 6.00 | g |
| Stearate | 1.50 | g |
| Brazil palm wax | 1.50 | g |
| Nipasol | 0.15 | g |

C phase

| | | |
|---|---|---|
| Purple coffee powder | 4.00 | g |
| Ivory nut powder red | 5.00 | g |
| Ivory nut powder purple | 5.00 | g |
| Ivory nut powder yellow | 2.00 | g |

D phase

| | | |
|---|---|---|
| Two poly propylene glycol carbonate | 10.00 | g |
| Isododecane | 24.00 | g |

E phase

| | | |
|---|---|---|
| Pearl powder 60325 | 6.00 | g |
| Pearl powder 7211VRB | 12.30 | g |

Ivory nut powder red was dyed with gardenia red.

Ivory nut powder yellow was dyed with gardenia yellow.

Ivory nut powder purple was dyed with purple sweet potato red.

### Example 14

Mannan in Deodorant Applications

Formulations:

Deodorant Creme:

| Phase | Raw Material | V1 (%) | V2 (%) | V3(%) |
|---|---|---|---|---|
| 1 | Water | 77.05 | 77.05 | 77.25 |
| 1 | Aluminum Chloride | 5 | 5 | 5 |
| 1 | Glycerine | 5 | 5 | 5 |
| 1 | Xanthan Gum | 1 | | 0.8 |
| 1 | Carbopol Ultrez 30 | | 1 | |
| 1 | Potassium Sorbate | 0.4 | 0.4 | 0.4 |
| 2 | Talc | 1 | 1 | |
| 2 | Mannan | | | 1 |
| 2 | Soy Oil | 5 | 5 | 5 |
| 2 | Emulsifier (Imwitor 372 P) | 5 | 5 | 5 |
| 2 | Beeswax | 0.5 | 0.5 | 0.5 |
| 3 | Sodium Hydroxide Solution | 0.05 | 0.05 | 0.05 |
| | Sum | 100 | 100 | 100 |

Phase 1. All ingredients were weighed into a beaker and heated to approximately 75° C. to about 80° C. until homogenous.

Phase 2. All ingredients were weighed into a beaker and heated to approximately 75° C. to about 80° C. until homogenous.

Phase 3. Phase 1 and Phase 2 compositions were added to each other and mixed until homogeneous.

Phase 4. The composition was cooled down to 30° C. and held there for approximately 5 to about 10 minutes.

Phase 5. To the cooled homogenous mixture from Phase 4 was added enough sodium hydroxide until a pH of about 3.5 to about 4 was achieved.

Results: V1: was too thick, less xanthan or other thickener should be used; V2: the Carbopol would not thicken with the high concentration of aluminum chloride; V3: had a good texture and no difference was noted if either mannan or talc was used.

Deodorant Roll On

| Phase | Raw Material | V1 (%) | V2 (%) |
|---|---|---|---|
| 1 | Water | 87.5 | 87.7 |
| 1 | Xanthan Gum | 1 | 0.8 |
| 2 | Aluminum Chloride | 10 | 10 |
| 3 | BioKons Neo | 1 | 1 |
| 4 | Talc | 0.5 | |
| 4 | Mannan | | 0.5 |
| | Sum | 100 | 100 |

Phase 1: Xanthan gum was added to water in incremental amounts until completely dissolved at a temperature from about 20° C. to about 25° C.

Phase 2: Aluminum chloride was added to the Phase 1 solution until completely dissolved at a temperature from about 20° C. to about 25° C.

Phase 3: The BioKons Neo, an essence, was added with stirring to the Phase 2 solution at a temperature from about 20° C. to about 25° C.

Phase 4: The remaining ingredients were added with stirring to the Phase 3 solution at a temperature from about 20° C. to about 25° C.

Conclusion:

There is no visible difference between formulations with Mannan or Talc.

Efficiency:

small panel test with 5 subjects

formulation with mannan and talc were tested against each other

| | Deodorant Creme | | Deodorant Roll On | |
|---|---|---|---|---|
| | with Mannan | with Talc | with Mannan | with Talc |
| | (V3) | (V1) | (V2) | (V1) |
| odour | ++++ | ++++ | +++++ | +++++ |
| prevention | | | | |

Conclusion:

The Roll-On-Formulation had a better performance than the Creme, but there was no difference between formulations with Talc or Mannan.

Stability:

the 4 formulations from the efficancy tests were stored at 40° C. for 24 h→no visible changes

Result:

Mannan can replace Talc in Deodorant Formulations.

Oil Absorption Value

Castor oil was been added with stirring to 1 gram powder until the powder became a sticky mass with no free flowing powder available.

Oil Absorption Value (g oil/g powder)

| | |
|---|---|
| Mannan | 1.28 |
| Titan Dioxide | 0.52 |
| Talc | 0.58 |
| Zinc Oxide | 0.36 |
| Magnesium Stearate | 0.56 |

Compact Powder Formulations:

| Sample | 1 | 2 | 3 | 4 | 5 | 6 | 7 |
|---|---|---|---|---|---|---|---|
| Talc | 6 | | 5 | 5 | 4.5 | | |
| Mannan | | 6 | | | | 4.5 | 4.5 |
| Magnesium Stearate | 0.5 | 0.5 | 1 | 1 | 1 | 1 | 1 |
| Titan Dioxide | | | | | 0.5 | 0.5 | |
| Color Pigments | 1 | 0.5 | 0.25 | 0.12 | 0.12 | 0.2 | 0.25 |
| Caprylic/Capric | 2.6 | 6.9 | 1.3 | 0.8 | 0.4 | 0.5 | 0.6 |
| Triglyceride | | | | | | | |
| Almond Oil | 0.1 | 0.1 | 0.1 | 0.1 | 0.1 | 0.1 | 0.1 |
| Soya Oil | 0.1 | 0.1 | 0.1 | 0.1 | 0.1 | 0.1 | 0.1 |
| Jojoba Oil | 0.1 | 0.1 | 0.1 | 0.1 | 0.1 | 0.1 | 0.1 |
| Tocopherol | | | | | | 0.005 | 0.005 |
| Preservative* | 0.1 | 0.1 | 0.1 | 0.1 | 0.1 | | |

*Propanediol, Phenethyl Alcohol, Undecyl Alcohol Tocopherol

Sample 1:

very dark, too much color pigments, to oily, texture more like cream than powder, hiding power not high enough.

Sample 2:

very dark, too much color pigments, to oily, texture more like cream than powder, hiding power better than sample 1.

Sample 3:

color a little bit too dark, good fixation on skin, hiding power not high enough.

Sample 4:

color ok for dark skin, a bit too oily, good fixation on skin, hiding power should be improved.

Sample 5:

color ok for fair skin, good texture, good fixation on skin, good hiding power.

Sample 6:

too bright, a bit too powdery, good fixation on skin, very good hiding power.

Sample 7:

color ok for fair skin, good texture, good fixation on skin, very good hiding power.

Oil Absorption Value

Mannan showed by far the best results. It absorbed more than the double amount of oil than the next best product talc. So it is suitable for applications like compact powders, mattifying creams or similar products were oil absorption is a desired value.

Compact Powder

Mannan shows a very good fixation on skin and also a very good hiding power. A lesser amount of Mannan than of Talc is needed if the same amount of oil is desired in a formulation (reverse it is possible to create formulations with a higher oil content if Mannan is used).

Mannan has a higher hiding power, so the use of Titan Dioxide isn't necessary to reach the same hiding power than a formulation with Talc+Titan Dioxide.

Formulations with a higher oil content appeared darker even if the same amount of pigments are used.

Higher amounts of Magnesium Stearate improve the fixation of the powder on the skin

The preservative utilized is approved for natural cosmetics but contains strong smelling components which gives the formulations a nice flowery touch. However some consumers might not like this kind of smell and because of the absence of water it isn't absolutely necessary to add a preservative. In this case only, Tocopherol should be added to increase the stability of the natural oils utilized.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A composition comprising a particulate ivory nut or particulate hydrolyzed or extracted ivory nut (mannan) material.

2. The composition of claim 1, wherein the particulate material has a particle size that is granular or powdered of less than 4000 microns, more particularly 3500 microns, 3000 microns, 2500 microns or 2000 microns (μm).

3. The composition of claim 2, wherein the particle size is less than 1500 microns.

4. The composition of claim 3, wherein the particle size is less than 1000 microns.

5. The composition of claim 4, wherein the particle size is less than 500 microns.

6. The composition of claim 5, wherein the particle size is less than 200 microns.

7. The composition of claim 6, wherein the particle size is less than 100 microns.

8. The composition of claim 7, wherein the particle size is less than 50 microns.

9. The composition of claim 8, wherein the particle size is less than 20 microns.

10. The composition of claim 9, wherein the particle size is less than 10 microns.

11. The composition of claim 10, wherein the particle size is less than 5 microns.

12. The composition of claim 11, wherein the particle size is less than 1 microns.

13. The composition of claim 12, wherein the particle size is less than 0.2 microns.

14. The composition of any of claim 1, wherein the particulate material is colored with a coloring agent.

15. The composition of claim 14, wherein the coloring agent is oil soluble, water soluble, or both.

16. The composition of claim 15, wherein the coloring agent is a natural organic material or an inorganic material.

17. The composition of claim 1, wherein the particulate is bleached.

18. The composition of claim 17, wherein the bleached particulate is white in color.

19. The composition of claim 1, wherein the particulate is coated.

20. The composition of claim 19, wherein the coating is an oil, an antioxidant, or a wax.

21. The composition of claim 1, further comprising a foodstuff, a toothpaste, a cosmetic composition, a rubber, a plastic, or a pharmaceutical agent.

22. The composition of claim 21, wherein the cosmetic composition is a baby powder, a pressed powder, lipstick, a body scrub, a soap, a lotion, a cream, a sun screen or a shampoo.

23. The composition of claim 21, wherein the toothpaste further comprises silicon dioxide, calcium carbonate, calcium hypophosphate, aluminum oxide, xanthan gum, sorbitol, menthol, ale powder, sodium benzoate, ethanol, holly oil, spearmint, water and combinations thereof.

24. The composition of claim 1, wherein the particulate is spherical, elliptical, irregular in form, shards, and can be amorphous or crystalline.

25. The composition of claim 1, wherein the particulate is used as a UV filter.

26. The composition of claim 21, wherein the percentage of the particulate within the composition is from 0.1% by weight to 99% by weight.

27. A composition comprising a mixture of talc and particulate ivory nut or talc and particulate hydrolyzed ivory nut (mannan) material or talc and extracted ivory nut (mannan) material wherein the mixture can be used as the composition of claim 21.

28. A composition comprising a mixture of titanium dioxide and particulate ivory nut or titanium dioxide and particulate hydrolyzed ivory nut (mannan) material or extracted ivory nut (mannan) material wherein the mixture can be used as the composition of claim 21.

29. A composition comprising a mixture of starch or modified starch and particulate ivory nut or starch or modified starch and particulate hydrolyzed ivory nut (mannan) material or starch or modified starch and extracted ivory nut (mannan) material wherein the mixture can be used as the composition of claim 21.

30. The composition of claim 27, wherein the percentage of the particulate ivory nut or particulate hydrolyzed ivory nut (mannan) material or extracted ivory nut (mannan) material of the composition is from 0.1% by weight to 99.9% by weight.
